SEQUENCE LISTING (5 sequences)

SEQ ID NO: 1 - protein, 738 aa, Artificial Sequence
  mouse antibody expression construct
MGWSWIFLFLLSGTAGVLSEVQLQQSGPVLVKPGASVKMSCKASGYTFTDYYMNWVKQSHGKSLEWIGVINPYNGDTSYNQKFKGKATLTVDKSSSTAYMELNSLTSEDSAVYYCARYYGSWFAYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKRGRKRRSGSGAPVKQTLNFDLLKLAGDVESNPGPMKLPVRLLVLMFWIPASSSDIVMTQTTLSLPVSLGDQASISCRSSQSIVHSNGNTYLEWYLQKPGQSPKLLIYKVSNRFSGVPDRFSGSGSGTDFTLKISRVEAEDLGVYYCFQGSHVPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 2 - protein, 704 aa, Artificial Sequence
  human antibody expression construct
MEFGLSWVFLVALLRGVQCQVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKMDMRVPAQLLGLLLLWLSGARCEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 776 aa, Artificial Sequence
  human antibody expression construct
MGWSWIFLFLLSGTAGVLSEVQLQQSGPVLVKPGASVKMSCKASGYTFTDYYMNWVKQSHGKSLEWIGVINPYNGDTSYNQKFKGKATLTVDKSSSTAYMELNSLTSEDSAVYYCARYYGSWFAYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGLDLDDVCAEAQDGELDGLWTTITIFISLFLLSVCYSASVTLFKVKSGSGAPVKQTLNFDLLKLAGDVESNPGPMKLPVRLLVLMFWIPASSSDIVMTQTTLSLPVSLGDQASISCRSSQSIVHSNGNTYLEWYLQKPGQSPKLLIYKVSNRFSGVPDRFSGSGSGTDFTLKISRVEAEDLGVYYCFQGSHVPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 4 - protein, 45 aa, Mus musculus
LDLDDVCAEAQDGELDGLWTTITIFISLFLLSVCYSASVTLFKVK

SEQ ID NO: 5 - protein, 45 aa, Homo sapiens
LQLEESCAEAQDGELDGLWTTITIFITLFLLSVCYSATITFFKVK

(19) United States
(12) Patent Application Publication
(10) Pub. No.: US 2024/0269272 A1
(43) Pub. Date: Aug. 15, 2024

(54) VACCINE COMPOSITION COMPRISING ENCODED ADJUVANT

(71) Applicant: NOUSCOM AG, Basel (CH)

(72) Inventors: Elisa SCARSELLI, Rome (IT); Alfredo NICOSIA, Rome (IT); Anna MORENA D'ALISE, Rome (IT); Armin LAHM, Rome (IT); Guido LEONI, Rome (IT); Emanuele SASSO, Pollena Trocchia (IT)

(21) Appl. No.: 18/566,077

(22) PCT Filed: Jun. 20, 2022
(86) PCT No.: PCT/EP2022/066733

§ 371 Date: Nov. 30, 2023

(30) Foreign Application Priority Data
Jun. 21, 2021 (EP) 21180661.7

(51) Int. Cl. A61K 39/39; A61K 39/00; A61P 35/00; C12N 15/86

(52) U.S. Cl. CPC A61K 39/39; A61K 39/0011; A61P 35/00; C12N 15/86; A61K 2039/53; A61K 2039/545; A61K 2039/55516; A61K 2039/55533; A61K 2039/55561; C12N 2710/10343

(57) ABSTRACT

The present invention relates to a vaccine composition comprising (1) a first set of one or more vectors comprising a nucleic acid encoding one or more adjuvants, wherein the first set of one or more vectors are adenoviral vectors, and (2) an antigen or a combination of antigens or a nucleic acid encoding said antigen or combination of antigens or a second set of one or more vectors comprising said nucleic acid. The invention further relates to said vaccine composition for use in the treatment or prophylaxis of a disease. In addition, the invention relates to a vaccine composition or vaccine kit for inducing an immune response comprising (1) a first nucleic acid encoding one or more adjuvants or a first set of one or more vectors comprising said first nucleic acid and (2) an antigen or a combination of antigens or a second nucleic acid encoding said second antigen or combination of antigens or a second set of one or more vectors comprising said second nucleic acid, wherein (1) is administered to a patient at a first location and (2) is administered to the patient at a second location, wherein the first location is the same or within 20 cm of the second location and the lymphatic system of the first and second location drains to the same lymph nodes. The invention also relates to a vaccination regimen comprising a first administration step comprising administration of an antigen and an encoded adjuvant, and a second administration step comprising administration of an antigen and/or an encoded adjuvant.

The present invention relates to a vaccine composition comprising an antigen or a combination of antigens and one or more encoded adjuvants. The invention further relates to such vaccine compositions for use in cancer therapy.

### BACKGROUND OF THE INVENTION

The field of vaccines is advancing rapidly with the aim of inducing a powerful immune response against a variety of infectious and neoplastic diseases. In this context, genetic cancer vaccines have the potential to become an important modality for cancer treatment in the coming years.

Cancer vaccines have to face the complexity of inducing a T cell response against tumor antigens that are either i) tumor associated antigens (TAAs) derived from a self-protein overexpressed in the tumor or ii) neo-antigens derived from a mutated self-protein. The most common genetic mutations in tumors are single nucleotide variants causing a single amino acid change flanked by the amino acid residues of the wild type protein. Most neo-antigens therefore contain an important “self” component and are considered weak immunogens. Overcoming immune tolerance to “self” is needed to obtain a strong immune response.

An adjuvant is an ingredient used in vaccines that helps to create a stronger immune response in people receiving the vaccine. However, some potent adjuvants are associated with severe side effects. Thus, there is a crucial need to optimize cancer vaccine potency while minimizing toxicity.

The present invention is based on the discovery that the immune response against an antigen can be significantly increased if the antigen is co-administered with an encoded adjuvant. Unexpectedly, the inventors found that the immune response against an antigen or combination of antigens is amplified when a vaccine composition comprises a set of one or more adenoviral vectors, preferably human adenoviral vectors encoding one or more adjuvants. Thus, the vaccine composition according to the invention provides inter alia for: (i) enhancing the immune response against an antigen or a combination of antigens; (ii) turning a suboptimal, weak immune response into a stronger immune response; (iii) enabling an immune response against antigens that otherwise do not produce any immune response; (iv) turning antigens from non-immunogenic into immunogenic; (v) enabling an immune response against TAAs; (vi) enabling an immune response against single antigens or combinations of only a small number of antigens, in particular TAAs or cancer neo-antigens; (vii) enabling a locally and temporally defined breakdown of immune tolerance against self antigens; (viii) enabling a limited systemic exposure of the adjuvant; (ix) enabling a limited unspecific activity of the adjuvant; (x) enabling a limited toxicity; (xi) enabling an easy co-formulation of antigen(s) and adjuvant(s); (xii) enabling simultaneous co-localized action of antigen(s) and adjuvant(s).

### SUMMARY OF THE INVENTION

In a first aspect, the present invention relates to a vaccine composition comprising (1) a first set of one or more vectors comprising a nucleic acid encoding one or more adjuvants, wherein the first set of one or more vectors are adenoviral vectors, and (2) an antigen or a combination of antigens or a nucleic acid encoding said antigen or combination of antigens or a second set of one or more vectors comprising said nucleic acid.

In a second aspect, the present invention relates to a vaccine composition according to the first aspect of the invention for use in the treatment or prophylaxis of a disease.

In a third aspect, the present invention relates to a vaccine composition or vaccine kit for inducing an immune response against an antigen or combination of antigens comprising (1) a first composition comprising a nucleic acid encoding one or more adjuvants or a first set of one or more vectors comprising said nucleic acid, and (2) a second composition comprising an antigen or a combination of antigens or a nucleic acid encoding said antigen or combination of antigens or a second set of one or more vectors comprising said nucleic acid, wherein (1) is administered to a patient at a first location and (2) is administered to the patient at a second location, wherein the first location is within 20 cm of the second location and the lymphatic system of the first location drains to the same lymph nodes as the lymphatic system of the second location or wherein the first location and the second location are the same.

In a fourth aspect, the present invention relates to a vaccination regimen comprising a first and a second administration step, wherein (a) the first administration step comprises administration of a vaccine composition according to the first, second or third aspect of the invention, and (b) the second administration step comprises administration of (1) a first composition comprising a nucleic acid encoding one or more adjuvants, or a first set of one or more vectors comprising said nucleic acid; and/or (2) a second composition comprising an antigen or a combination of antigens, or a nucleic acid encoding said antigen or combination of antigens, or a second set of one or more vectors comprising said nucleic acid.

### DETAILED DESCRIPTION OF THE INVENTION

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention, which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, Leuenberger, H. G. W, Nagel, B. and Kolbl, H. eds. (1995), Helvetica Chimica Acta, CH-4010 Basel, Switzerland) and as described in “Pharmaceutical Substances: Syntheses, Patents, Applications” by Axel Kleemann and Jurgen Engel, Thieme Medical Publishing, 1999; the “Merck Index: An Encyclopedia of Chemicals, Drugs, and Biologicals”, edited by Susan Budavari et al., CRC Press, 1996, and the United States Pharmacopeia-25/National Formulary-20, published by the United States Pharmcopeial Convention, Inc., Rockville Md., 2001.

Throughout this specification and the claims which follow, unless the context requires otherwise, the word “comprise”, and variations such as “comprises” and “comprising”, will be understood to imply the inclusion of a stated feature, integer or step or group of features, integers or steps but not the exclusion of any other feature, integer or step or group of integers or steps. In the following passages different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

### Definitions

In the following, some definitions of terms frequently used in this specification are provided. These terms will, in each instance of its use, in the remainder of the specification have the respectively defined meaning and preferred meanings.

The terms “polynucleotide” and “nucleic acid” are used interchangeably herein and are understood as a polymeric or oligomeric macromolecule made from nucleotide monomers. Nucleotide monomers are composed of a nucleobase, a five-carbon sugar (such as but not limited to ribose or 2′-deoxyribose), and one to three phosphate groups. Typically, a nucleic acid is formed through phosphodiester bonds between the individual nucleotide monomers. In the context of the present invention preferred nucleic acid molecules include but are not limited to ribonucleic acid (RNA), modified RNA, deoxyribonucleic acid (DNA), and mixtures thereof such as e.g. RNA-DNA hybrids. The nucleic acids, can e.g. be synthesized chemically, e.g. in accordance with the phosphotriester method (see, for example, Uhlmann, E. & Peyman, A. (1990) Chemical Reviews, 90, 543-584).

As used herein, the term “protein”, “peptide”, “polypeptide”, “peptides” and “polypeptides” are used interchangeably throughout. These terms are used in the context of the present invention to refer to both naturally occurring peptides, e.g. naturally occurring proteins and synthesized peptides that may include naturally or non-naturally occurring amino acids.

The term “immune response” in the context of the present invention includes cellular and humoral immune response.

The term “antigen” is used in the context of the present invention to refer to any structure recognized by molecules of the immune response, e.g. antibodies, T cell receptors (TCRs) and the like. Preferred antigens are cellular proteins or fragments thereof that are associated with a particular disease. Antigens are recognized by highly variable antigen receptors (B-cell receptor or T-cell receptor) of the adaptive immune system and may elicit a humoral or cellular immune response. Antigens that elicit such a response are also referred to as “immunogens”. A fraction of the proteins inside cells, irrespective of whether they are foreign or cellular, are processed into smaller peptides and presented to by the major histocompatibility complex (MHC).

The term “vector” as used in the present invention refers to a polynucleotide or a mixture of a polynucleotide and proteins capable of introducing foreign genetic material, in particular DNA or RNA, into a cell, preferably a mammalian cell, where it can be replicated and/or expressed. Examples of vectors include but are not limited to plasmids, cosmids, phages, viruses or artificial chromosomes. Expression vectors may contain “replicon” polynucleotide sequences that facilitate the autonomous replication of the expression vector in a host cell. Once in the host cell, the expression vector may replicate independently of or coincidental with the host chromosomal DNA, and several copies of the vector and its inserted DNA can be generated. In case that replication incompetent expression vectors are used—which is often the case for safety reasons—the vector may not replicate but merely direct expression of the nucleic acid. Depending on the type of expression vector the expression vector may be lost from the cell, i.e. only transiently expresses the antigens or adjuvants encoded by the nucleic acid or may be stable in the cell. Expression vectors typically contain expression cassettes, i.e. the necessary elements that permit transcription of the nucleic acid into an mRNA molecule.

The terms “adenoviral vector” and “adenovector” are used interchangeably throughout this application.

The term “adeno-associated virus” (AAV) refers to a virus belonging to the family of Parvoviridae, containing several genera which can be subdivided into the family of Parvovirinae comprising Parvovirus, Erythrovirus, Dependovirus, Amdovirus and Bocavirus and the family of Densoviriniae comprising Densovirus, Iteravirus, Brevidensovirus, Pefudensovirus and Contravirus. The unique life cycle of AAV and its ability to infect both non-dividing and dividing cells with persistent expression have makes it an attractive vector. An additional attractive feature of the wild-type AAV virus is the lack of apparent pathogenicity.

The terms “adeno-associated virus vector” or “AAV vector” are used interchangeably throughout this application.

Vaccine compositions as described in the present invention include an antigen or a combination of antigens or a nucleic acid encoding said antigen or combination of antigens or one or more vectors comprising said nucleic acid. The vaccine compositions further comprise one or more encoded adjuvants, and may additionally include stabilizers, further adjuvants, antibiotics, and preservatives.

In the context of a vaccine composition according to the present invention, the term “antigen” refers to one or more proteins or fragments thereof delivered to a subject to induce an immune response. The antigen may be delivered either in the form of a protein or may be encoded, wherein the nucleic acid encoding the antigen may or may not be comprised in a vector.

The term “adjuvant” is used in the context of the present invention to refer to agents that augment, stimulate, activate, potentiate, or modulate the immune response to the antigen comprised in the vaccine composition. Examples of such adjuvants include, but are not limited to, cytokines, cytokine analogues, cytokine receptors, modulators of a checkpoint molecule, synthetic polynucleotide adjuvants (e.g. polyarginine or polylysine), activators of interferon (IFN) genes, antagonists of indoleamide 2,3-dioxygenase (IDO), adenosine deaminase (ADA) or proliferator-activated receptor gamma coactivator 1-alpha (PGC-1). Preferred adjuvants are selected from the group consisting of an agonist of OX40, preferably OX40L, an agonist of ICOS, preferably ICOSL, an agonist of CD40, preferably CD40L and an antagonistic CTLA-4 specific antibody or antibody like protein. In the context of the present invention, a vaccine composition comprises one or more encoded adjuvants. Thus, in the context of a vaccine composition according to the present invention, the term adjuvant refers to an encoded adjuvant. In the vaccine composition of the first aspect of the invention, the one or more adjuvants are encoded by a nucleic acid that is comprised in an adenoviral vector, preferably a human adenoviral vector. In the vaccine composition or vaccine kit for use of the third aspect of the invention and the vaccination regiment of the fourth aspect of the invention, the delivery of the one or more encoded adjuvants is not limited to viral vectors.

The skilled person is well aware of different suitable ways to deliver encoded antigens and/or adjuvants. Delivery can be achieved e.g. by DNA, in particular plasmid DNA; RNA, in particular in vitro transcribed (IVT) RNA, non-replicating messenger RNA and/or self-amplifying RNA (SAM); a viral vector; an alphavirus vector, a venezuelan equine encephalitis (VEE) virus vector, a sindbis (SIN) virus vector, a semliki forest virus (SFV) virus vector, also preferably a replication competent or incompetent adenoviral vector a poxvirus vector, a vaccinia virus vector or a modified vaccinia ankara (MVA) vector, a simian or human cytomegalovirus (CMV) vector, a lymphocyte choriomeningitis virus (LCMV) vector, a retroviral or lentiviral vector.

In instances where antigen or adjuvant is encoded by RNA, administration is either achieved as naked nucleic acid or in a complex with a carrier. The RNA may also be administered in combination with stabilizing substances such as RNase inhibitors. Carriers useful according to the invention include, for example, lipid-containing carriers such as cationic lipids, liposomes, micelles, lipid nanoparticles and lipid-polymer hybrid nanoparticles. A preferred carrier for the administration of RNA is a lipid nanoparticle or a lipid-polymer hybrid nanoparticle. A typical lipid nanoparticle formulation is composed of pH-responsive lipids or cationic lipids bearing tertiary or quaternary amines to encapsulate the polyanionic mRNA; neutral helper lipids such as zwitterionic lipids [i.e., 1,2-dioleoyl-sn-glycero-3-phosphoethanolamine (DOPE) or 1,2-distearoyl-sn-glycero-3-phosphocholine (DSPC)] and/or sterol lipids (i.e., cholesterol) to stabilize the lipid bilayer of the lipid nanoparticle and to enhance mRNA delivery efficiency; and a polyethylene glycol (PEG)-lipid to improve the colloidal stability in biological environments by reducing aspecific absorption of plasma proteins and forming a hydration layer over the nanoparticles. Lipid-polymer hybrid nanoparticles consist of a biodegradable mRNA-loaded polymer core coated with a lipid layer. Usually, the lipid envelope is organized into a lipid bilayer or lipid monolayer containing a mixture of cationic or ionizable lipids, helper lipids, and pegylated lipids (Guevara et al., 2020, Advances in Lipid Nanoparticles for mRNA-Based Cancer Immunotherapy. Front. Chem. 8:589-959).

The term “immunomodulator” refers to a compound selected from the group consisting of a modulator of a checkpoint molecule and a cytokine or cytokine analogue. In the context of the present invention, an immunomodulator may be administered in combination with the vaccine composition of the invention, either prior to or after the vaccine composition or simultaneously. Thus, the immunomodulator, if present, is a further component of the vaccine composition in addition to the adjuvant. The immunomodulator is preferably administered as a protein, wherein the adjuvant is encoded. Preferred immunomodulators are selected from the group consisting of an antagonistic CTLA-4 specific antibody or antibody like protein, an antagonistic PD-1 specific antibody or antibody like protein, and IL-2 or an analogue thereof.

The term “antibody” is used in the context of the present invention to refer to a glycoprotein belonging to the immunoglobulin superfamily. An antibody refers to a protein molecule that can be produced by plasma cells and is used by the immune system to identify and neutralize foreign objects such as bacteria and viruses. The antibody recognizes a unique part of the foreign target, its antigen. The term “antibody” refers to a molecule having the overall structure of an antibody, for example an IgG antibody. When referring to IgG in general, IgG1, IgG2, IgG3 and IgG4 are included, unless defined otherwise. IgG antibody molecules are Y-shaped molecules comprising four polypeptide chains: two heavy chains and two light chains. Each light chain consists of two domains, the N-terminal domain being known as the variable or VL domain (or region) and the C-terminal domain being known as the constant (or CL) domain (constant kappa (Cx) or constant lambda (CX) domain). Each heavy chain consists of four domains. The N-terminal domain of the heavy chain is known as the variable (or VH) domain (or region), which is followed by the first constant domain (CH1), the hinge region, and then the second and third constant domains (CH2 and CH3). In an assembled antibody, the VL and VH domains associate to form an antigen binding site. Also, the CL and CH1 domains associate to keep one heavy chain associated with one light chain. The two heavy-light chain heterodimers associate by interaction of the CH2 and CH3 domains and interaction between the hinge regions of the two heavy chains. The term “antibody” as used herein also includes molecules which may have chimeric domain replacements (i.e. at least one domain replaced by a domain from a different antibody), such as an IgG1 antibody comprising an IgG3 domain (e.g. the CH3 domain of IgG3). Further, the term generally refers to multispecific, e.g. bispecific or trispecific antibodies. The term antibody also includes molecules carrying one or more mutations within the heavy chain constant domain.

The term “antibody like-molecule” as used within the context of the present specification comprises antibody derivatives and antibody mimetics.

The term “antibody mimetic” refers to compounds, which can specifically bind antigens, similar to an antibody, but are not structurally related to antibodies. Usually, antibody mimetics are artificial peptides or proteins with a molar mass of about 3 to 20 kDa which comprise one, two or more exposed domains specifically binding to an antigen. Typically, such an antibody mimetic comprises at least one variable peptide loop attached at both ends to a protein scaffold. This double structural constraint greatly increases the binding affinity of the antibody-like protein to levels comparable to that of an antibody. The length of the variable peptide loop typically consists of 10 to 20 amino acids. The scaffold protein may be any protein having good solubility properties. Preferably, the scaffold protein is a small globular protein. Examples include inter alia the LACI-D1 (lipoprotein-associated coagulation inhibitor); affilins, e.g. human-γ B crystalline or human ubiquitin; cystatin; Sac7D from Sulfolobus acidocaldarius; lipocalin and anticalins derived from lipocalins; DARPins (designed ankyrin repeat domains); SH3 domain of Fyn; Kunitz domain of protease inhibitors; monobodies, e.g. the 10th type III domain of fibronectin; adnectins: knottins (cysteine knot miniproteins); atrimers; evibodies, e.g. CTLA4-based binders, affibodies, e.g. three-helix bundle from Z-domain of protein A from Staphylococcus aureus; Trans-bodies, e.g. human transferrin; tetranectins, e.g. monomeric or trimeric human C-type lectin domain; microbodies, e.g. trypsin-inhibitor-II; affilins; armadillo repeat proteins. Nucleic acids and small molecules are sometimes considered antibody mimetics as well (aptamers), but not artificial antibodies, antibody fragments and fusion proteins composed from these. Common advantages over antibodies are better solubility, tissue penetration, stability towards heat and enzymes, and comparatively low production costs.

The term “binding” according to the invention preferably relates to a specific binding. The term “binding affinity” generally refers to the strength of the sum total of noncovalent interactions between a single binding site of a molecule (e.g., an antibody) and its binding partner (e.g., target or antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). “Specific binding” means that a binding moiety (e.g. an antibody) binds stronger to a target such as an epitope for which it is specific compared to the binding to another target. A binding moiety binds stronger to a first target compared to a second target if it binds to the first target with a dissociation constant (Kd) which is lower than the dissociation constant for the second target. The dissociation constant (Kd) for the target to which the binding moiety binds specifically is more than 10-fold, preferably more than 20-fold, more preferably more than 50-fold, even more preferably more than 100-fold, 200-fold, 500-fold or 1000-fold lower than the dissociation constant (Kd) for the target to which the binding moiety does not bind specifically.

Accordingly, the term “Kd” (measured in “mol/L”, sometimes abbreviated as “M”) is intended to refer to the dissociation equilibrium constant of the particular interaction between a binding moiety (e.g. an antibody or fragment thereof) and a target molecule (e.g. an antigen or epitope thereof). Affinity can be measured by common methods known in the art, including but not limited to surface plasmon resonance based assay (such as the BIAcore assay); quartz crystal microbalance assays (such as Attana assay); enzyme-linked immunoabsorbent assay (ELISA); and competition assays (e.g. RIA's). Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention.

Typically, antibodies or antibody mimetics bind to their target with a sufficient binding affinity, for example, with a Kd value of between 500 nM-1 pM, i.e. about 500 nM, about 450 nM, about 400 nM, about 350 nM, about 300 nM, about 250 nM, about 200 nM, about 150 nM, about 100 nM, about 50 nM, about 10 nM, about 1 nM, about 900 pM, about 800 pM, about 700 pM, about 600 pM, about 500 pM, about 400 pM, about 300 pM, about 200 pM, about 100 pM, about 50 pM, or about 1 pM, such as 500 nM, 450 nM, 400 nM, 350 nM, 300 nM, 250 nM, 200 nM, 150 nM, 100 nM, 50 nM, 10 nM, 1 nM, 900 pM, 800 pM, 700 pM, 600 pM, 500 pM, 400 pM, 300 pM, 200 pM, 100 pM, 50 pM or 1 pM.

The term “immunoglobulin (Ig)” as used herein refers to immunity conferring glycoproteins of the immunoglobulin superfamily. “Surface immunoglobulins” are attached to the membrane of e.g. effector cells or endothelial cells by their transmembrane region and encompass molecules such as but not limited to neonatal Fc-receptor, B-cell receptors, T-cell receptors, class I and II major histocompatibility complex (MHC) proteins, beta-2 microglobulin (β2M), CD3, CD4 and CD8.

The term “antibody derivative” as used herein refers to a molecule comprising at least the domains it is specified to comprise, but not having the overall structure of an antibody such as IgA, IgD, IgE, IgG, IgM, IgY or IgW, although still being capable of binding a target molecule. Said derivatives may be, but are not limited to functional (i.e. target binding, particularly specific target binding) antibody fragments or combinations thereof. It also relates to an antibody to which further antibody domains have been added, such as further variable domains. Thus, the term antibody derivative also includes multispecific (bispecific, trispecific, tetraspecific, pentaspecific hexaspecific etc.) and multivalent (bivalent, trivalent, tetravalent etc.) antibodies.

Bispecific antibodies occur in a plurality of formats (Brinkmann and Kontermann, Mabs 2017, Vol. 9, No. 2, 182-212). Examples for bispecific antibodies consisting only of antigen binding domains are bivalent Fabs (bi-Fabs). Another example are formats comprising only variable domains (Fv) but no constant domains. Formats comprising only variable domains have the advantage of a very low molecular weight leading to a good tumor penetrance, which is important for oncologic applications. Due to the lack of a constant domain, which mediates binding to the FcRn, such formats have a reduced plasma half-life.

The term “epitope”, also known as antigenic determinant, is used in the context of the present invention to refer to the segment of an antigen, preferably peptide that is bound by molecules of the immune system, e.g. B-cell receptors, T-cell receptors or antibodies. The epitopes bound by antibodies or B cells are referred to as “B cell epitopes” and the epitopes bound by T cells are referred to as “T cell epitopes”. In this context, the term “binding” preferably relates to a specific binding, which is defined as a binding with an association constant between the antibody or T cell receptor (TCR) and the respective epitope of 1×10⁵ M-1 or higher, preferably of 1×10⁶ M-1, 1 15×10⁷ M-1, 1×10⁸ M-1 or higher. The skilled person is well aware how to determine the association constant (see e.g. Caoili, S. E. (2012) Advances in Bioinformatics Vol. 2012). Preferably, the specific binding of antibodies to an epitope is mediated by the Fab (fragment, antigen binding) region of the antibody, specific binding of a B-cell is mediated by the Fab region of the antibody comprised by the B-cell receptor and specific binding of a T-cell is mediated by the variable (V) region of the T-cell receptor. T cell epitopes are presented on the surface of an antigen presenting cell, where they are bound to Major Histocompatibility (MHC) molecules. There are at least two different classes of MHC molecules termed MHC class I, II respectively. Epitopes presented through the MIC-I pathway elicit a response by cytotoxic T lymphocytes (CD8+ cells), while epitopes presented through the MHC-II pathway elicit a response by T-helper cells (CD4+ cells).

T cell epitopes presented by MHC Class I molecules are typically peptides between 8 and 12 amino acids in length and T cell epitopes presented by MHC Class II molecules are typically peptides between 13 and 17 amino acids in length. MHC Class III molecules also present non-peptidic epitopes as glycolipids. Accordingly, the term “T cell epitope” preferably refers to a 8 to 11 or 13 to 17 amino acid long peptide that can be presented by either a MHC Class I or MHC Class II molecule. Epitopes usually consist of chemically active surface groupings of amino acids, which may or may not carry sugar side chains and usually have specific three-dimensional structural characteristics, as well as specific charge characteristics. Conformational and non-conformational epitopes are distinguished in that the binding to the former but not the latter is lost in the presence of denaturing solvents.

In the context of the present invention, the terms “CTLA4 specific antibody” and “anti-CTLA4 antibody” are used interchangeably.

In the context of the present invention, the term “antagonistic antibody” refers to an antibody that is capable of inhibiting a biological activity of the molecule it binds to. If the antagonistic antibody binds to a certain receptor, it is capable of blocking or dampening the signaling pathway downstream of the receptor or competing with the receptor ligand. The skilled person is well aware that the determination of an antagonistic activity depends on multiple parameters, e.g. the assay or the cell type used. In the context of the present invention, an antagonistic antibody specific for CTLA-4 is characterized by the following activity: removal of the negative signaling of T-cell responses mediated by CTLA4, i.e. removal of the inhibitory effect of CTLA4 signaling on T cell activation, resulting therefore in an enhanced immune response.

In the context of the present invention, the term “agonistic antibody” refers to an antibody that binds to a receptor and activates the signaling pathway downstream of the receptor in a way comparable to the receptor ligand. An example for an agonistic antibody is CP-870,893, which binds to and activates the receptor CD40. The skilled person is well aware that the determination of an agonistic activity depends on multiple parameters, e.g. the assay or the cell type used.

In the context of the present invention, the term “agonist ligand” refers to a soluble ligand that binds to a receptor and activates the signaling pathway downstream of the receptor. An example for an agonist ligand is OX40L, which binds to and activates the receptor OX40.

The term “tumor associated antigen (TAA)” is used in the context of the present invention to refer to an antigen derived from a self-protein overexpressed in a tumor, i.e. a protein that is expressed not at all or only at low levels in healthy tissue and at increased levels in tumor tissue. A TAA may be a full length protein or a fragment thereof.

The term Cancer Testis (CT) antigens refers to a group of proteins united by their importance in development and in cancer immunotherapy. In general, expression of these proteins is restricted to male germ cells in the adult animal. However, in cancer these developmental antigens are often re-expressed. Thus, they represent a category of tumor associated antigens. CT antigens have been described in several tumors including melanoma, liver cancer, lung cancer, bladder cancer, and pediatric tumors such as neuroblastoma. A regularly updated list of CT antigens can be found at http://www.cta.lncc.br/index.php. Important CT antigens in cancer therapy include MAGE-A1, MAGE-A3, MAGE-A4, NY-ESO-1, PRAME, CT83 and SSX2.

The term “neo-antigen” is used in the context of the present invention to refer to an antigen not present in normal/germline cells but which occurs in transformed, in particular cancerous cells. A neo-antigen may comprise one or more, e.g. 2, 3, 4, 5 or more neo-epitopes. It is preferred that the length of each neo-antigen included in the antigen of the present invention is selected in such a way as to ascertain that there is a low likelihood of comprising epitopes that occur in normal/germline cells. Typically, this can be ascertained in that the neo-antigen comprises 12 or less amino acids C-terminally and/or N-terminally of the amino acid change(s) that created a neo-epitope.

The mutated cancer protein comprising the neo-antigen is generated by a mutation occurring at the level of the DNA and wherein the mutated protein can comprise
a) one or more single aa changes caused by one or more point mutations representing non-synonymous single nucleotide variations (SNVs); and/or
b) a non-wildtype amino acid sequence caused by insertions/deletions resulting in a frame-shift peptide or an in-frame insertion of one or more non-wildtype amino acids or deletion of one or more wildtype amino acids; and/or
c) a non-wildtype amino acid sequence caused by alteration of exon boundaries or by mutations generating intron retention; and/or
d) a mutated cancer protein generated by a gene fusion event.

A neo-antigen that is the result of one or more single amino acid changes caused by a genomic non-synonymous SNV point mutation is referred to in the context of the present invention as a single amino acid mutant peptide.

The term “frame-shift peptide” is used in the context of the present invention to refer to the complete non wild-type translation product of the protein-encoding segment of a nucleic acid comprising an insertion or deletion mutations causing a shift of the Open Reading Frame (ORF).

The term “open reading frame” abbreviated “ORF” is used in the context of the present invention to refer to a sequence of nucleotides that can be translated into a consecutive string of amino acids. Typically, an ORF contains a start codon, a subsequent region usually having a length which is a multiple of 3 nucleotides, but does not contain a stop codon (TAG, TAA, TGA, UAG, UAA, or UGA) in the given reading frame. An ORF codes for a protein where the amino acids into which it can be translated form a peptide-linked chain.

A neo-antigen that is the result of a non-wildtype amino acid sequence caused by alteration of exon boundaries or by mutations generating intron retention is referred to in the context of the present invention as a splice site mutant peptide.

A neo-antigen that is the result of a mutated cancer protein generated by a gene fusion event is referred to in the context of the present invention as a read-through mutation peptide.

The term “cytokine analogue” is used in the context of the present invention to refer to a cytokine that has been modified to exhibit improved physicochemical characteristics such as being more robust, having favorable pharmacokinetic properties, having an enhanced half-life, being more amenable to certain delivery systems and formulations or having an enhanced or more selective biological activity. The cytokine analogue may comprise amino acid changes compared to the unmodified cytokine or may comprise posttranslational modifications, e.g. PEGylation.

The term “expression cassette” is used in the context of the present invention to refer to a nucleic acid molecule, which comprises at least one nucleic acid sequence that is to be expressed, e.g. a nucleic acid encoding the antigens of the present invention or a part thereof, operably linked to transcription and translation control sequences. Preferably, an expression cassette includes cis-regulating elements for efficient expression of a given gene, such as promoter, initiation-site and/or polyadenylation-site. Preferably, an expression cassette contains all the additional elements required for the expression of the nucleic acid in the cell of a patient. A typical expression cassette thus contains a promoter operatively linked to the nucleic acid sequence to be expressed and signals required for efficient polyadenylation of the transcript, ribosome binding sites, and translation termination. Additional elements of the cassette may include, for example, enhancers or intron elements. An expression cassette preferably also contains a transcription termination region downstream of the encoded antigen to provide for efficient termination. The termination region may be obtained from the same gene as the promoter sequence or may be obtained from a different gene.

The term “operably linked” as used in the context of the present invention refers to an arrangement of elements, wherein the components so described are configured so as to perform their usual function. A nucleic acid is “operably linked” when it is placed into a functional relationship with another nucleic acid sequence. For example, a promoter is operably linked to one or more transgenes, if it affects the transcription of the one or more transgenes. Further, control elements operably linked to a coding sequence are capable of effecting the expression of the coding sequence. The control elements need not be contiguous with the coding sequence, so long as they function to direct the expression thereof. Thus, for example, intervening untranslated yet transcribed sequences can be present between a promoter sequence and the coding sequence and the promoter sequence can still be considered “operably linked” to the coding sequence.

The term “pharmaceutical preparation” or “pharmaceutical composition” as used in the context of the present invention is intended to include the vaccine composition according to the invention, i.e. an antigen or a combination of antigens (protein or encoded), one or more adjuvants (protein or encoded), optionally an immunomodulator and a pharmaceutically acceptable carrier and/or excipient.

“Pharmaceutically acceptable” as used in the context of the present invention means approved by a regulatory agency of the Federal or a state government or listed in the U.S. Pharmacopeia or other generally recognized pharmacopeia for use in animals, and more particularly in humans.

The term “pharmaceutically acceptable carrier”, as used herein, refers to a pharmacologically inactive substance such as but not limited to a diluent, excipient, surfactants, stabilizers, physiological buffer solutions or vehicles with which the therapeutically active ingredient is administered. Such pharmaceutical carriers can be liquid or solid. Liquid carrier include but are not limited to sterile liquids, such as saline solutions in water and oils, including but not limited to those of petroleum, animal, vegetable or synthetic origin, such as peanut oil, soybean oil, mineral oil, sesame oil and the like. Saline solutions and aqueous dextrose and glycerol solutions can also be employed as liquid carriers, particularly for injectable solutions. A saline solution is a preferred carrier when the pharmaceutical composition is administered intravenously. Examples of suitable pharmaceutical carriers are described in “Remington's Pharmaceutical Sciences” by E. W. Martin.

Suitable pharmaceutical “excipients” include starch, glucose, lactose, sucrose, gelatine, malt, rice, flour, chalk, silica gel, sodium stearate, glycerol monostearate, talc, sodium chloride, dried skim milk, glycerol, propylene, glycol, water, ethanol and the like.

“Surfactants” include anionic, cationic, and non-ionic surfactants such as but not limited to sodium deoxycholate, sodium dodecylsulfate, Triton X-100, and polysorbates such as polysorbate 20, polysorbate 40, polysorbate 60, polysorbate 65 and polysorbate 80.

“Stabilizers” include but are not limited to mannitol, sucrose, trehalose, albumin, as well as protease and/or nuclease antagonists.

“Physiological buffer solution” that may be used in the context of the present invention include but are not limited to sodium chloride solution, demineralized water, as well as suitable organic or inorganic buffer solutions such as but not limited to phosphate buffer, citrate buffer, tris buffer (tris(hydroxymethyl)aminomethane), HEPES buffer ([4 (2 hydroxyethyl)piperazino]ethanesulphonic acid) or MOPS buffer (3 morpholino-1 propanesulphonic acid). The choice of the respective buffer in general depends on the desired buffer molarity. Phosphate buffer are suitable, for example, for injection and infusion solutions.

An “effective amount” or “therapeutically effective amount” is an amount of a therapeutic agent sufficient to achieve the intended purpose. The effective amount of a given therapeutic agent will vary with factors such as the nature of the agent, the route of administration, the size and species of the animal to receive the therapeutic agent, and the purpose of the administration. The effective amount in each individual case may be determined empirically by a skilled artisan according to established methods in the art.

As used herein, “treat”, “treating”, “treatment” or “therapy” of a disease or disorder means accomplishing one or more of the following: (a) reducing the severity of the disorder; (b) limiting or preventing development of symptoms characteristic of the disorder(s) being treated; (c) inhibiting worsening of symptoms characteristic of the disorder(s) being treated; (d) limiting or preventing recurrence of the disorder(s) in an individual that has previously had the disorder(s); and (e) limiting or preventing recurrence of symptoms in individuals that were previously symptomatic for the disorder(s).

### Aspects of the Invention and Preferred Embodiments

In a first aspect, the present invention relates to a vaccine composition comprising (1) a first set of one or more vectors comprising a nucleic acid encoding one or more adjuvants, wherein the first set of one or more vectors are adenoviral vectors, and (2) an antigen or a combination of antigens or a nucleic acid encoding said antigen or combination of antigens or a second set of one or more vectors comprising said nucleic acid.

### Vectors

The first set of vectors are preferably human adenoviral vectors, more preferably replication incompetent human adenoviral vectors. It is preferred that the first set of vectors are group C human adenoviral vectors. Group C (also referred to a species C) of human adenoviruses comprises hAd1, hAd2, hAd5, hAd6 and hAd57. In preferred embodiments, the first set of vectors are selected from the group consisting of hAd6, hAd57 and hAd5. In some embodiments, the first set of vectors are selected from of hAd6 and hAd5. Preferably, the first set of vectors are selected from hAd6 and hAd57, more preferably hAd6.

In instances where the antigen or combination of antigens is encoded, the antigen or combination of antigens is encoded by a nucleic acid that is not comprised in the first set of one or more vectors.

It is preferred that the vaccine composition comprises a second set of one or more vectors comprising a nucleic acid encoding the antigen or combination of antigens. It can be envisioned that the second set of vectors are adenoviral or adeno-associated viral (AAV) vectors.

The one or more adjuvants are encoded by a nucleic acid comprised in the first set of one or more vectors, wherein the antigen or combination of antigens (if encoded by a nucleic acid comprised in a vector) are comprised in the second set of one or more vectors. In other words, the antigen is not encoded by a nucleic acid that is comprised in the first set of one or more vectors.

Preferably, the second set of vectors are replication competent or incompetent adenoviral vectors, preferably replication incompetent. It is preferred that the adenoviral vectors are derived from great apes, preferably non-human great apes. Preferred non-human great apes from which the adenoviruses are derived are Chimpanzee (Pan), preferably Bonobo (Pan paniscus) and common Chimpanzee (Pan troglodytes), Gorilla (Gorilla) and Orangutan (Pongo). In preferred embodiments, the second set of vectors are adenoviral vectors derived from chimpanzee or bonobo or gorilla, most preferably derived from gorilla. Typically, naturally occurring non-human great ape adenoviruses are isolated from stool samples of the respective great ape.

The most preferred vectors are non-replicating adenoviral vectors based on gorilla adenoviral vectors.

Other suitable vectors are non-replicating adenoviral vectors based on hAd4, hAd5, hAd6, hAd7, hAd11, hAd26, hAd35, hAd49, hAd57, ChAd3, ChAd4, ChAd5, ChAd6, ChAd7, ChAd8, ChAd9, ChAd10, ChAd11, ChAd16, ChAd17, ChAd19, ChAd20, ChAd22, ChAd24, ChAd26, ChAd30, ChAd31, ChAd37, ChAd38, ChAd44, ChAd55, ChAd63, ChAd73, ChAd82, ChAd83, ChAd146, ChAd147, PanAd1, PanAd2, and PanAd3 vectors or replication-competent Ad4 and Ad7 vectors. The human adenoviruses hAd4, hAd5, hAd6, hAd7, hAd11, hAd26, hAd35, hAd49 and hAd57 are well known in the art. Vectors based on naturally occurring ChAd3, ChAd4, ChAd5, ChAd6, ChAd7, ChAd8, ChAd9, ChAd10, ChAdl1, ChAd16, ChAd17, ChAd19, ChAd20, ChAd22, ChAd24, ChAd26, ChAd30, ChAd31, ChAd37, ChAd38, ChAd44, ChAd63 and ChAd82 are described in detail in WO 2005/071093. Vectors based on naturally occurring PanAd1, PanAd2, PanAd3, ChAd55, ChAd73, ChAd83, ChAd146, and ChAd147 are described in detail in WO 2010/086189.

Preferred AAV vectors are based on AAV-serotypes selected from the group consisting of AAV-1, AAV-2, AAV-2-AAV-3 hybrid, AAV-3a, AAV-3b, AAV-4, AAV-5, AAV-6, AAV-6.2, AAV-7, AAV-8, AAV-9, AAV-10, AAVrh.10, AAV-11, AAV-12, AAV-13 and AAVrh32.33.

### Antigens

The antigen or the combination of antigens may be delivered either in the form of proteins or may be encoded by nucleic acids. The nucleic acids may or may not be comprised in a vector. In some embodiments, the antigen or the combination of antigens is encoded by RNA and delivered by a lipid nanoparticle or a lipid-polymer hybrid nanoparticle. It is preferred that the antigen or the combination of antigens is encoded by a nucleic acid that is comprised in a second set of vectors.

In preferred embodiments of all aspects of the invention, the antigen or the combination of antigens is selected from a cancer antigen, a viral antigen, a bacterial antigen and a fungal antigen or the combination of antigens comprises one or more antigens selected from the group consisting of a cancer antigen, a viral antigen, a bacterial antigen and a fungal antigen.

In preferred embodiments of all aspects of the invention, the antigen or the combination of antigens elicits no or only a suboptimal immune response in a subject in the absence of the one or more adjuvants, encoded by the human adenoviral vectors of the first set of vectors. In other words, in preferred embodiments, the antigen or the combination of antigens is a weak antigen, i.e. an antigen having low immunogenicity. Factors influencing the immunogenicity of an antigen are its foreignness (it must be recognizable as non-self), its molecular size, its chemical composition and heterogeneity and its ability to be presented in a complex with an MHC molecule on the surface of a cell. As mentioned above, tumor associated antigens and tumor neoantigens are often weak antigens. A suboptimal immune response may also be referred to as “weak immune response”. The skilled person is well aware of methods to quantify an immune response and to decide whether an immune response is to be classified as “suboptimal” or even “absent”. In particular, the immune response is quantified by analysing the T cell response to an antigen or a combination of antigens. Activation of T cells in response to an antigen or a combination of antigens can be analysed by determination of cytokine secretion, in particular secretion of IFNγ, IL-2, TNF-alpha, IL-4, IL-5, and/or IL-13. In preferred embodiments, the immune response is quantified by determination of the number of T cells producing IFNγ per 10⁶ splenocytes in response to an antigen or a combination of antigens. An exemplary assay that may be used in the determination of the immune response is the IFN-γ ELISpot assay described in Example 11. The humoral immune response can be analysed by measuring the serum antibody levels against an antigen.

A “suboptimal” immune response is preferably defined as less than 600, less than 500, less than 400, less than 300, less than 200, most preferably less than 150 IFNγ producing T cells per 10⁶ splenocytes.

An “absent” immune response is preferably defined as less than 100, less than 60, less than 40, more preferably less than 30, IFNγ producing T cells per 10⁶ splenocytes.

The inventors found that in instances where administration of an antigen or a combination of antigens (alone or together with a systemically administered, non-encoded adjuvant, i.e. a protein adjuvant) resulted in a “suboptimal” immune response, co-administration of one or more adenoviral vector encoded adjuvants together with the same antigen or the same combination of antigens resulted in a significant increase of the immune response, in particular in increase to a response no longer classified as “suboptimal” (FIG. 3, FIG. 8. FIG. 9).

In addition, the inventors found that in instances where administration of an antigen or a combination of antigens (alone or together with a systemically administered, non-encoded adjuvant) produced essentially no immune response (i.e. an “absent” immune response), co-administration of one or more adenoviral vector encoded adjuvants together with the same antigen or the same combination of antigens resulted in the generation of an immune response (FIG. 4, FIG. 6, FIG. 7).

The inventors further found that in instances where administration of an antigen or a combination of antigens (alone or together with a systemically administered, non-encoded adjuvant) resulted in an adequate immune response (i.e. an immune response stronger than an immune response classified as “suboptimal”), co-administration of one or more adenoviral vector encoded adjuvants together with the same antigen or the same combination of antigens resulted in an even stronger immune response.

Surprisingly, the inventors found that the described effects varied with the type of adenovirus used for encoding the adjuvant. Human adenoviral vectors, in particular human group C adenoviral vectors resulted in higher levels of adjuvant (FIG. 1A) and an increased immune response (FIG. 1i). Adenoviral vectors hAd5, hAd6 and hAd57 (which has a very high sequence similarity to hAd6) were found to be particularly advantageous.

The inventors also showed that providing an encoded adjuvant, preferably in a human adenoviral vector, leads to reduced systemic exposure compared to the same adjuvant administered as a protein (FIG. 5). This demonstrates an increased safety of an encoded adjuvant, in particular an adjuvant encoded in an adenoviral vector. Without wishing to be bound by theory, the inventors propose that the adenoviral vectors, particularly human adenoviral vectors, more particularly human group C adenoviral vectors, more particularly hAd5, hAd6 and hAd57, even more particularly hAd6 and hAd57, and most particularly hAd6, generate sufficiently high local levels of adjuvant such that the immune response is increased, without concomitant high systemic levels of adjuvant.

In preferred embodiments, the antigen or the combination of antigens comprises or consists of one or more cancer antigens selected from tumor associated antigens (TAAs), and/or cancer neo-antigens.

In preferred embodiments, the TAAs are specific for a defined tumor type, in particular bladder cancer, head and neck cancer, non small cell lung cancer (NSCLC), melanoma, thymoma, colon cancer; breast cancer, ovarian cancer, liver cancer; or kidney cancer. In some embodiments, the TAAs are characterized by i.e. a protein that is expressed not at all or only at low levels in healthy tissue and at increased levels in tumor tissue. A common class of TAAs are for example Cancer Testis (CT) antigens. In general, expression of these proteins is restricted to male germ cells in the adult animal. However, in cancer these developmental antigens are often re-expressed.

In preferred embodiments, the cancer neo-antigens are selected from the group consisting of a single amino acid mutant peptide, a frame-shift peptide, an intron read-through mutation peptide, and a splice site mutant peptide. In some embodiments, the cancer neo-antigens are fragments of cancer tissue expressed mutated proteins wherein the fragment comprises a central non-wt amino acid caused by a mutation (one or more non-synonymous single nucleotide variants) flanked on both sides by the respective wildtype amino acid sequence, preferably 12 amino acids on both sides. In some embodiments, the cancer neo-antigens can contain more than one non-wild type amino acid.

Similarly, the nucleic acid encoding a combination of antigens may present in a single vector or may be distributed between more than one vector of the second set of vectors. Single antigens may be joined head to tail with or without linkers. If present, linkers between antigens or between groups of antigens can be derived from naturally-occurring multi-domain proteins or can be generated by design. Linkers include flexible linkers and/or in vivo cleavable linkers that can be processed by cellular proteases. Suitable linker sequences are well known in the art and preferably comprise or consist of between 1 to 10 amino acids. Linkers preferably consist or comprise small amino acids like Ser and Gly.

In preferred embodiments of all aspects of the invention, the second set of vectors comprises a nucleic acid encoding at least 1, at least 3, at least 5, at least 8, at least 10, at least 20, at least 30, at least 40, at least 50 TAAs.

In preferred embodiments of all aspects of the invention, the second set of vectors comprises a nucleic acid encoding at least 5, at least 10, at least 20, at least 30, at least 40, at least 50, at least 100 cancer neo-antigens.

Generally, the prophylactic or therapeutic vaccination against viral, bacterial or fungal infection does not require as many different antigens to be effective as the vaccination in the therapy of proliferative diseases. Nevertheless, there are some viruses like, e.g. HIV that have a large epitope diversity, in particular in the coat proteins. To elicit a broad immune response multiple antigens can be included. In preferred embodiments of all aspects of the invention, the second set of vectors comprises a nucleic acid encoding at least 1, at least 3, at least 5, at least 8, at least 10, at least 20, at least 30, at least 40, at least 50, at least 100 viral, bacterial or fungal antigens.

A vaccine composition that comprises more antigens usually elicits a stronger immune response than a vaccine composition comprising only few antigens, wherein “few antigens” refers to 10 antigens or less, in particular 5 antigens or less.

### Adjuvant

The vaccine composition according to the first aspect of the invention may comprise one encoded adjuvant or several encoded adjuvants. The nucleic acid encoding the one or more adjuvants may present in a single vector or may be distributed between more than one vector of the first set of vectors. E.g. if the adjuvant is an antibody, the heavy chain may be encoded in one vector and the light chain may be encoded in another vector, or heavy and light chain may be encoded in the same vector. If more than one encoded adjuvant is present, they may be comprised in a single vector or in a set of vectors.

In all aspects of the invention, the one or more encoded adjuvants may be membrane-bound or soluble. The skilled person is aware that a membrane-bound adjuvant is encoded by a nucleic acid comprising a transmembrane domain and an ER sorting signal. In preferred embodiments of all aspects of the invention, the one or more adjuvant is selected from a modulator of a checkpoint molecule, a cytokine, preferably selected from IL-2, IL-1β, IL-7, IL-15, IL-18, GM-CFS, and INF-γ, or a cytokine analogue, a cytokine receptor, preferably CD25 (IL-2 alpha receptor), a synthetic polynucleotide adjuvant, a poly-amino acid adjuvant, preferably polyarginine or polylysine, an activator of interferon genes, preferably STING (Stimulator of interferon genes; also known as MITA and MPYS), adenosine deaminase (ADA) or proliferator-activated receptor gamma coactivator 1-alpha (PGC-1α). In preferred embodiments, the modulator of a checkpoint molecule is selected from the group consisting of an agonist of a tumor necrosis factor (TNF) receptor superfamily member or an agonist of a B7-CD28 superfamily member, wherein preferably the agonist is a (soluble) ligand or an agonistic antibody or antibody like protein (e.g. CP-870,893 for CD40); and an antagonist of PD-1, PD-L1, A2AR, B7-H3 (e.g. MGA271), B7-H4, BTLA, CTLA-4, IDO, KIR, LAG3, TIM-3, TIGIT or VISTA, wherein preferably the antagonist is an antagonistic antibody or antibody like protein. In preferred embodiments, the agonist of a TNF receptor superfamily member is CD27, CD40 (e.g. CP-870,893), OX40, GITR or CD137. In preferred embodiments, the agonist of a B7-CD28 superfamily member is CD28 or ICOS.

In preferred embodiments of all aspects of the invention, the one or more adjuvants are selected from the group consisting of an agonist of OX40, preferably OX40L, an agonist of ICOS, preferably ICOSL, an agonist of CD40, preferably CD40L, and an antagonistic CTLA-4 specific antibody or antibody like protein, wherein the antagonistic CTLA-4 specific antibody or antibody like protein may be soluble or may comprise a transmembrane domain and an ER sorting signal, i.e. a membrane bound antibody. In some embodiments, the transmembrane domain is a murine transmembrane domain according to SEQ ID NO: 4. In preferred embodiments, the transmembrane domain is a human transmembrane domain according to SEQ ID NO: 5.

In preferred embodiments of all aspects of the invention, the one or more adjuvants are
(1) an antagonistic CTLA-4 specific antibody or antibody like protein;
(2) an agonist of OX40, preferably OX40L;
(3) an agonist of ICOS, preferably ICOSL;
(4) an agonist of CD40, preferably CD40L;
(5) an antagonistic CTLA-4 specific antibody or antibody like protein and an agonist of OX40, preferably OX40L;
(6) an antagonistic CTLA-4 specific antibody or antibody like protein and an agonist of ICOS, preferably ICOSL;
(7) an antagonistic CTLA-4 specific antibody or antibody like protein and an agonist of CD40, preferably CD40L;
(8) an agonist of OX40, preferably OX40L, and an agonist of ICOS, preferably ICOSL;
(9) an agonist of OX40, preferably OX40L, and an agonist of CD40, preferably CD40L; or
(10) an agonist of ICOS, preferably ICOSL and an agonist of CD40, preferably CD40L.

In preferred embodiments of all aspects of the invention, the antagonistic CTLA-4 specific antibody is Ipilimumab.

In preferred embodiments of all aspects of the invention, the one or more adjuvants comprise a transmembrane domain and an ER sorting signal. In other words, when the encoded adjuvant is expressed, it is a membrane-bound protein.

CTLA-4 receptor molecule expression and function is intrinsically linked with T-cell activation. CTLA4 is immediately upregulated following T-cell receptor (TCR) engagement (signal 1), with its expression peaking 2-3 days after activation. CTLA4 dampens TCR signaling competing with the costimulatory molecule CD28 for binding to the B7 ligands B7-1 (CD80) and B7-2 (CD86), for which CTLA4 has higher avidity and affinity. Because both B7-1 and B7-2 provide positive costimulatory signals through CD28 (signal 2) to T cells engaged with TCR (signal 1), inhibition of the interaction of both molecules with CTLA4 is therefore necessary. Anti-CTLA-4 antibodies blocking CTLA-4's inhibitory activity therefore potentiate T cell activation. An anti-CTLA4 antibody (Ipilimumab; BMS) has been successfully developed for cancer immunotherapy based on the induction of long-lasting protection for some melanoma patients. However, the therapeutic potential of systemic delivery of anti-CTLA-4 antibodies is limited by significant immunotherapy-related adverse effects.

OX40L in the context of the present invention refers to OX40 ligand (human OX40: NP_003317, murine OX40L: NP_033478). OX40L is the ligand for OX40 (also known as CD134 or TNFRSF4) and is stably expressed on many antigen-presenting cells such as DC2s (a subtype of dendritic cells), macrophages, and activated B lymphocytes. The binding of OX40L to OX40 is a source of survival signal for T cells and enables the development of memory T cells.

ICOSL in the context of the present invention refers to ICOS ligand (human ICOSL: NP_056074, murine ICOSL: NP_056605).

CD40L in the context of the present invention refers to CD40 ligand (human CD40L: NP_000065, murine CD40L: NP_035746).

In the examples, murine versions of ICOSL, CD40L and OX40L were used.

In some embodiments, the adjuvant is an antibody encoded as one contiguous amino acid sequence comprising a 2A sequence, which allows the generation of the separate heavy and light chains. In some embodiments, the adjuvant is an antibody encoded as one contiguous amino acid sequence containing a first signal peptide, the heavy chain, a furin site, a 2A sequence, a second signal peptide and the light chain. Such constructs were used for Ipilimumab and 9D9 in the examples section.

The inventors demonstrated that the adjuvant activity of the encoded adjuvant is significantly superior to that of the protein adjuvant delivered systemically by intraperitoneal injection (FIG. 3). Without wishing to be bound by any theory, these results indicate that co-administration of adenoviral vector encoded adjuvant ensures timely co-localization of the antigen and the adjuvant for effective adjuvanticity. In addition, the inventors demonstrated that the concentration of the adjuvant in the serum is significantly reduced for the encoded adjuvant compared to the protein adjuvant injected subcutaneously or intraperitoneally (FIG. 5). Thus, the adjuvant effect of the encoded adjuvant is achieved with very limited systemic exposure.

All terms used with respect to the following aspects of the invention have the meanings as defined with respect to the first aspect of the invention, unless specifically defined otherwise. Further, all embodiments specified for the first aspect that are applicable to the other aspects are also envisaged for those aspects, unless specifically defined otherwise.

In a second aspect, the present invention relates to a vaccine composition according to the first aspect of the invention for use in the treatment or prophylaxis of a disease.

In preferred embodiments, the vaccine composition is for use in treating a proliferative disease in a subject. Preferably, the proliferative disease is cancer and/or a tumor.

It is generally preferred that the tumor is at least of stage Tis or T1 (excluding Tx and T0), preferably of at least stage T2, T3 or T4. It may at the same time be of all stages N (e.g. Nx or N0) and M (e.g. M0), and in a preferred embodiment at least of stage N1, N2 or N3 and/or M1). This refers to the TNM classification, which defines the tumor stages as follows:

T: size or direct extent of the primary tumor

Tx: tumor cannot be assessed

Tis: carcinoma in situ

T0: no evidence of tumor

T1, T2, T3, T4: evidence of primary tumor, size and/or extension increasing with stage

N: degree of spread to regional lymph nodes

Nx: lymph nodes cannot be assessed

N0: no regional lymph nodes metastasis

N1: regional lymph node metastasis present; at some sites, tumor spread to closest or small number of regional lymph nodes

N2: tumor spread to an extent between N1 and N3 (N2 is not used at all sites)

N3: tumor spread to more distant or numerous regional lymph nodes (N3 is not used at all sites)

M: presence of distant metastasis

M0: no distant metastasis

M1: metastasis to distant organs (beyond regional lymph nodes)

Exemplary stages envisaged to benefit in particular from the invention are Tis and any of N (preferably N1 or N2 or N3) and any of M (preferably M1), T1 and any of N (preferably N1 or N2 or N3) and any of M (preferably M1), T2 and any of N (preferably N1 or N2 or N3) and any of M (preferably M1), T3 and any of N (preferably N1 or N2 or N3) and any of M (preferably M1), and T4 and any of N (preferably N1 or N2 or N3) and any of M (preferably M1). The presence of a tumor and its spread in a patient can be detected using imaging methods, for example Computed Tomography (CT) scans, Magnetic Resonance Imaging (MRI), isotopic diagnostics with radioactive tracers that are detected by scintigraphy in Positron Emission Tomography (PET) or a combination thereof. Imaging methods can also be combined with other methods like for example ultra sound examination, endoscopic examination, mammography, biomarker detection in the blood, fine needle biopsy or a combination thereof. The size of tumors that can be detected by imaging methods depends on the method used and is about 1.5 cm in diameter for isotope imaging, about 3 mm in diameter for CT and MRI and about 7 mm in diameter for PET-based methods (Erdi. (2012) Molecular Imaging and Radionuclide Therapy 21(1): 23).

Preferably, the presence of a tumor (“evidence”) determined with a method selected from the group consisting of detection of circulating tumor cell free DNA, Computed Tomography (CT) scan, Magnetic Resonance Imaging (MRI), isotopic diagnostics with radioactive tracers that are detected by scintigraphy in Positron Emission Tomography (PET), and any combination of the foregoing. In one embodiment, one or more of the foregoing methods or combination thereof is a combined with a method of the group consisting of ultra sound examination, endoscopic examination, mammography, biomarker detection in the blood, fine needle biopsy and any combination of the foregoing.

In preferred embodiments of the second aspect, the cancer is selected from the group consisting of malignant neoplasms of lip, oral cavity, pharynx, a digestive organ, respiratory organ, intrathoracic organ, bone, articular cartilage, skin, mesothelial tissue, soft tissue, breast, female genital organs, male genital organs, urinary tract, brain and other parts of central nervous system, thyroid gland, endocrine glands, lymphoid tissue, and hematopoietic tissue.

Generally, it is preferred that the subject has a tumor at a TNM stage as described above.

In one embodiment, the tumor is characterized by a lesion of at least about 3 mm in diameter, preferably at least 7 mm in diameter, and more preferably at least 1.5 cm in diameter.

In preferred embodiments, the vaccine composition is administered in combination with one or more immunomodulators, more particularly with anti-PD1. The one or more immunomodulators, in particular the anti-PD1 are preferably administered as a protein.

It can be envisioned that the administration of the one or more immunomodulators is initiated before initiation of the administration of the vaccine composition, or after initiation of the administration of the vaccine composition, or administration of the one or more immunomodulators is initiated simultaneously with the initiation of the administration of the vaccine composition.

In another embodiment of the second aspect of the invention, the vaccine composition is provided for the treatment of an infectious disease, such as a viral, bacterial or fungal infection.

In a third aspect, the present invention relates to a vaccine composition or vaccine kit for inducing an immune response against an antigen or combination of antigens comprising (1) a first composition comprising a nucleic acid encoding one or more adjuvants or a first set of one or more vectors comprising said nucleic acid, and (2) a second composition comprising an antigen or a combination of antigens or a nucleic acid encoding an antigen or a combination of antigens or a second set of one or more vectors comprising said nucleic acid, wherein (1) is administered to a patient at a first location and (2) is administered to the patient at a second location, wherein the first location is within 20 cm of the second location and the lymphatic system of the first location drains to the same lymph nodes as the lymphatic system of the second location or wherein the first location and the second location are the same.

In the context of the present invention, the expression “inducing an immune response” refers to a cellular immune response and/or a humoral immune response as described herein. In some embodiments, the vaccine composition or vaccine kit is provided for use in the treatment or prophylaxis of a disease, preferably for use in treating or preventing a proliferative disease or an infectious disease, more preferably cancer.

The antigen or combination of antigens may be delivered either in the form of a protein or may be encoded, wherein the nucleic acid encoding the antigen or combination of antigens may or may not be comprised in a vector. The one or more adjuvants are encoded, wherein the nucleic acid encoding the one or more adjuvants (i.e. the first nucleic acid) may or may not be comprised in a vector. The nucleic acid encoding the one or more adjuvants (i.e. the first nucleic acid) may be one molecule or more than one, such as two or more nucleic acid molecules. The skilled person is aware that the terms “one nucleic acid molecule”, “two nucleic acid molecules” etc. are not meant to indicate absolute numbers of nucleic acid molecules, but to indicate the amount of different nucleic acid molecules, i.e. nucleic acid molecules having a different sequence. In instance where the adjuvant is an antibody, the heavy chain may be encoded by one nucleic acid molecule, and the light chain may be encoded by another nucleic acid molecule, or heavy and light chain may be encoded by one nucleic acid molecule. If more than one encoded adjuvant is present, they may be encoded by one nucleic acid molecule or several nucleic acid molecules. Similarly, the nucleic acid encoding the one or more adjuvants (i.e. the first nucleic acid) may present in a single vector or may be distributed between more than one vector of the first set of vectors. E.g. if the adjuvant is an antibody, the heavy chain may be encoded in one vector and the light chain may be encoded in another vector, or heavy and light chain may be encoded in the same vector. If more than one encoded adjuvant is present, they may be comprised in a single vector or in a set of vectors. In some embodiments of the third aspect of the invention, the one or more adjuvants and/or the antigen or combination of antigens are encoded by RNA and delivered by a lipid nanoparticle or a lipid-polymer hybrid nanoparticle. In preferred embodiments of the third aspect of the invention, the one or more adjuvants and/or the antigen or combination of antigens are encoded by nucleic acids comprised in a first set of vectors and a second set of vectors, respectively. Most preferably, the vectors are those described for the first aspect of the invention.

Surprisingly, the inventors found that the effect of the encoded adjuvant is lost if antigen and adjuvant are administered at distant locations, wherein the lymphatic system of both locations do not drain to the same lymph nodes, such as contralateral extremities (FIG. 2). Without wishing to be bound by theory, the inventors assume that for effective adjuvanticity, it is important that antigen and adjuvant act simultaneously and in close proximity, in particular within one lymph node. This can be achieved by using an encoded adjuvant, preferably an adenoviral vector encoded adjuvant, more preferably a human adenoviral vector encoded adjuvant. In addition, the simultaneous action in close proximity is enhanced if antigen, preferably encoded antigen and encoded adjuvant, are either administered as a mixture or at close locations (draining to the same lymph node) and within a short time interval. If both antigen and adjuvant are encoded, the nucleic acid sequences encoding the antigen and the adjuvant are not comprised within the same molecule, e.g. not in the same vector or on the same RNA molecule. The vaccine composition or vaccine kit of the third aspect of the invention may comprise the first and second composition as a mixture or as two separate components. In other words, the vaccine composition or vaccine kit may be formulated for simultaneous or separate administration of the first and second composition. The components of the first composition and the second composition may be comprised within one composition, but are separate molecules. The first and the second nucleic acid are not comprised in the same nucleic acid molecule. The first set of one or more vectors and the second set of one or more vectors are different sets of vectors. Thus, antigen and adjuvant are delivered as separate molecules, but these separate molecules are delivered in temporal and spatial proximity.

In preferred embodiments, the first location is within 17.5 cm, 15 cm, 12.5 cm, 10 cm, 7.5 cm, 5 cm, 2.5 cm, 1 cm, 0.5 cm, 0.25 cm or 0.1 cm of the second location. In most preferred embodiments, the first location and the second location are the same. The skilled person is aware that in instances where (1) and (2) are administered as a mixture, the first location and the second location are identical and there is no time interval between the administration of (1) and (2).

It can be envisioned that (1) and (2) are administered by intramuscular, subcutaneous, intradermal, intra-peritoneal or intra-pleural injection. In preferred embodiments, (1) and (2) are administered by the same route, e.g. both are administered by intramuscular injection. However, as long as (1) are administered to a first and a second tissue wherein lymphatic system of the first and the second tissue drains to the same lymph nodes, the administration does not necessarily have to be by the same route.

In preferred embodiments of the third aspect of the invention, (1) and (2), i.e. the encoded adjuvant and the antigen (protein or encoded) are administered within a time interval of 30 min or less, 20 min or less, 15 min or less, 10 min or less, 5 min or less, 3 min or less, or 1 min or less. In most preferred embodiments of the third aspect of the invention, (1) and (2) are administered to the patient as a mixture, i.e. (1) and (2) are administered simultaneously and at the same location.

Furthermore, simultaneous action in close proximity can be enhanced by use of an encoded adjuvant comprising a transmembrane domain and an ER sorting signal. When expressed, such adjuvants are membrane bound. Unlike soluble adjuvants, they cannot diffuse, but are linked to the cell by which they are expressed, thereby facilitating the action in close proximity. In addition, membrane bound adjuvants only exert a local effect and therefore limit undesired effects due to systemic exposure of the soluble adjuvant. Thus, in preferred embodiments, the adjuvant comprises a transmembrane domain and an ER sorting signal. Examples for membrane bound adjuvants are OX40L, CD40L, ICOSL or membrane-bound versions of anti-CTLA4.

The vaccine composition or vaccine kit for inducing an immune response against an antigen or combination of antigens is preferably for use in treating a disease in a subject. The disease may be an infectious disease or a proliferative disease, preferably a proliferative disease. Preferably, the proliferative disease is cancer and/or a tumor.

In preferred embodiments of the second and third aspect, viral vectors, in particular adenoviral vectors comprising a nucleic acid encoding one or more adjuvants are administered to a subject, in particular a human subject, preferably by intramuscular administration, at a viral particle load (vp) of 10\hat{ }vp or more, 2×10\hat{ }vp or more, 4×10\hat{ }vp or more, and 10\hat{ }11 vp or less, 8×10\hat{ }vp or less, 6×10\hat{ }vp or less, and adenoviral vectors encoding an antigen or a combination of antigens are administered to a subject, in particular a human subject, preferably by intramuscular administration, at a viral particle load (vp) of 5×10\hat{ }vp or more, 6×10\hat{ }vp or more, 7×10\hat{ }vp or more, 8×10\hat{ }vp or more, and 2×10\hat{ }11 vp or less, 10\hat{ }11 vp or less, 9×10\hat{ }10 vp or less.

In a fourth aspect, the present invention relates to a vaccination regimen comprising a first and a second administration step, wherein (a) the first administration step comprises administration of a vaccine composition according to the first, second or third aspect of the invention, and (b) the second administration step comprises administration of (1) a first composition comprising a nucleic acid encoding one or more adjuvants, or a first set of one or more vectors comprising said nucleic acid; and/or (2) a second composition comprising an antigen or a combination of antigens, or a nucleic acid encoding an antigen or a combination of antigens, or a second set of one or more vectors comprising said nucleic acid.

The vaccine composition administered in the first administration step may be the vaccine composition provided by the first aspect of the invention. The vaccine composition administered in the first administration step may also be the vaccine composition provided for use by the second or third aspect of the invention.

The one or more encoded adjuvants administered in the first and second administration step may be the same or different, preferably the same. They may be selected from the adjuvants described with respect to the first aspect of the invention. In preferred embodiments, the one or more adjuvants comprised in the first and second vaccine composition are selected from the group consisting of an agonist of OX40, preferably OX40L, an agonist of ICOS, preferably ICOSL, an agonist of CD40, preferably CD40L, and an antagonistic CTLA-4 specific antibody or antibody like protein, wherein the antagonistic CTLA-4 specific antibody or antibody like protein may be soluble or may comprise a transmembrane domain and an ER sorting signal, i.e. a membrane bound antibody.

If an antigen or a combination of antigens is administered in the second administration step (protein or encoded), the antigen or combination of antigens are the same as in the first administration step.

In instances where the second administration step comprises administration of an antigen (protein or encoded), the administration can be described as prime boost regimen.

In some embodiments, the vaccination regimen is a heterologous prime boost regimen with two different viral vectors. In such embodiments, the first and second administration are preferably separated by an interval of at least 1 week, preferably of 6 weeks.

It is preferred that both the first and the second administration step comprise administration of a first set of one or more vectors comprising a nucleic acid encoding one or more adjuvants. In other words, both the first and the second administration step comprise administration of one or more encoded adjuvants, wherein the encoded adjuvants are comprised in vectors. In addition, it is preferred both the first and the second administration step comprise administration of a second set of one or more vectors comprising a nucleic acid encoding the antigen or combination of antigens. In other words, both the first and the second administration step comprise administration of an encoded antigen or combination of antigens, wherein the encoded antigen or combination of antigens are comprised in vectors.

The first and second set of vectors of the second administration step may be viral vectors selected from the group consisting of an alphavirus vector, a venezuelan equine encephalitis (VEE) virus vector, a sindbis (SIN) virus vector, a semliki forest virus (SFV) virus vector, a simian or human cytomegalovirus (CMV) vector, a lymphocyte choriomeningitis virus (LCMV) vector, a retroviral or lentiviral vector, an adenoviral vector, an AAV vector, a poxvirus vector, a vaccinia virus vector or a modified vaccinia ankara (MVA) vector.

It is preferred that the first set of vectors (“adjuvant vectors”) of the first and second administration step are adenoviral vectors. More preferably, they are human adenoviral vectors, preferably selected from those described for the first set of vectors of the first aspect of the invention. In preferred embodiments, the first set of vectors of the first administration step are different adenoviral vectors, preferably different human adenoviral vectors, than the first set of vectors of the second administration step.

It is further preferred that the second set of vectors (“antigen vectors”) of the first and second administration step are selected from those described for the second set ofvectors of the first aspect of the invention. However, the second set of vectors of the first administration step is different from the second set of vectors of the first administration step. In other words, the second set of vectors (“antigen vectors”) of the first and second administration step are different vectors, but comprise the same antigen or combination of antigens.

In preferred embodiments of the first administration step, the first set of vectors (“adjuvant vectors”) are human adenoviral vectors and the second set of vectors (“antigen vectors”) are adenoviral vectors.

In preferred embodiments of the second administration step, the first set of vectors (“adjuvant vectors”) are adenoviral vectors, AAV vectors or MVA vectors, preferably adenoviral vectors, and the second set of vectors (“antigen vectors”) are MVA vectors.

Surprisingly, the inventors found that re-administration of the adjuvant alone, preferably adenoviral vector encoded adjuvant, enhances the antitumor efficacy of a neoantigen vaccine (FIG. 10). Thus, in some embodiments the second administration step comprises administration of the adjuvant, preferably an adenoviral vector encoded adjuvant, more preferably a human adenoviral vector encoded adjuvant, but not of the antigen. In such embodiments, the first set of vectors are preferably the same in the first and second administration step (i.e., the same adjuvant in the same vector). In addition, in such embodiments, the first and second administration steps are preferably separated by an interval of about 1 day. Preferably, the first and the second administration are via the same route.

In preferred embodiments of the vaccination regimen, the first and/or the second administration step further comprises administration of at least one immunomodulator.

In another aspect, the present invention relates to a pharmaceutical preparation or pharmaceutical composition comprising a vaccine composition according to the first aspect and a pharmaceutically acceptable carrier and/or excipient. The pharmaceutical preparation or composition may further comprise at least one immunomodulator. The invention also relates to said pharmaceutical preparation or composition for use in preventing or treating, in particular treating, a proliferative disease in a subject.

For preparing pharmaceutical compositions of the present invention, pharmaceutically acceptable carriers can be either solid or liquid. Solid form compositions include powders, tablets, pills, capsules, lozenges, cachets, suppositories, and dispersible granules. A solid excipient can be one or more substances, which may also act as diluents, flavouring agents, binders, preservatives, tablet disintegrating agents, or an encapsulating material. In powders, the excipient is preferably a finely divided solid, which is in a mixture with the finely divided inhibitor of the present invention. In tablets, the active ingredient is mixed with the carrier having the necessary binding properties in suitable proportions and compacted in the shape and size desired. Suitable excipients are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. For preparing suppositories, a low melting wax, such as a mixture of fatty acid glycerides or cocoa butter, is first melted and the active component is dispersed homogeneously therein, as by stirring. The molten homogeneous mixture is then poured into convenient sized moulds, allowed to cool, and thereby to solidify. Tablets, powders, capsules, pills, cachets, and lozenges can be used as solid dosage forms suitable for oral administration.

Liquid form compositions include solutions, suspensions, and emulsions, for example, water, saline solutions, aqueous dextrose, glycerol solutions or water/propylene glycol solutions. For parenteral injections (e.g. intravenous, intraarterial, intraosseous infusion, intramuscular, subcutaneous, intraperitoneal, intradermal, and intrathecal injections), liquid preparations can be formulated in solution in, e.g. aqueous polyethylene glycol solution. A saline solution is a preferred carrier when the pharmaceutical composition is administered intravenously.

Preferably, the pharmaceutical composition is in unit dosage form. In such form the composition may be subdivided into unit doses containing appropriate quantities of the active component. The unit dosage form can be a packaged composition, the package containing discrete quantities of the composition, such as packaged tablets, capsules, and powders in vials or ampoules. Also, the unit dosage form can be a capsule, an injection vial, a tablet, a cachet, or a lozenge itself, or it can be the appropriate number of any of these in packaged form.

The composition, if desired, can also contain minor amounts of wetting or emulsifying agents, or pH buffering agents.

Furthermore, such pharmaceutical composition may also comprise other pharmacologically active substances.

In another aspect, the present invention relates to a vaccination kit comprising in separate packaging (i) a vaccine composition according to the first aspect; and (ii) at least one immunomodulator.

In the context of the present specification, the adjuvant is an encoded adjuvant, wherein the immunomodulator is preferably a protein.

In yet another aspect, the present invention relates to a method of treating or preventing a proliferative or infective disease, preferably a proliferative disease, comprising administration of an effective amount of the vaccine composition of the first aspect of the invention or the vaccine composition as described with respect to the third aspect of the invention, to a patient in need thereof.

In preferred embodiments of any of the above aspects, the one or more immunomodulators are a cytokine selected from IL-2, IL-1β, IL-7, IL-12, IL-15, IL-18, GM-CFS, and INF-γ, a cytokine analogue selected from analogues of IL-2, IL-1β, IL-7, IL-12, IL-15, IL-18, GM-CFS, and INF-γ or a modulator of a checkpoint molecule selected from the group consisting of an agonist of a tumor necrosis factor (TNF) receptor superfamily member, an agonist of a B7-CD28 superfamily member; and an antagonist of PD-1, PD-L1, A2AR, B7-H3 (e.g. MGA271), B7-H4, BTLA, CTLA-4, IDO, KIR, LAG3, TIM-3, or VISTA.

In preferred embodiments of any of the above aspects, the at least one immunomodulator is selected from an antagonistic CTLA-4 specific antibody or antibody like protein, an antagonistic PD-1 specific antibody or antibody like protein, and/or IL-2 or an analogue thereof. The one or more immunomodulators are preferably administered as a protein.

### FIGURE LEGENDS

FIG. 1 A) Serum concentration of the anti-mCTLA4 (clone 9d9) in mice receiving Ad6, Ad5, GAd20 and ChAd68 vectors encoding the 9D9 anti-mCTLA4 (10\hat{ }8 viral particles, vp), measured 7 days after injection. B) Effect of encoded anti-CTLA4 on the vaccine-induced T cell response. C57Bl6 mice were vaccinated im with a GAd vaccine encoding seven CD8 T cell neo-antigens selected from the MC38 tumor model (vaccine, dose of 2×10\hat{ }7 vp) administered alone or in combination (as mixture) with Ads encoding anti-mCTLA4 (Ad6, Ad5, GAd20 and ChAd68 vectors encoding the 9D9 anti-mCTLA4; 10\hat{ }8 vp each), Shown are the total responses (number of T cells producing IFNγ per millions of splenocytes) to the CD8 epitopes encoded in the vaccine measured in the 5 experimental groups by an IFN-γ ELISpot assay.

FIG. 2 shows the effect of encoded anti-mCTLA4 antibody (Ad-9d9) on the vaccine-induced T cell response when co-administered with the vaccine as a mixture in one anatomical site (mix), when given as separate nearby administrations (5 min time difference) at the same anatomical site as the mixture (separate) or at two distant sites (contralateral). C57Bl6 mice were vaccinated im with a GAd vaccine encoding seven CD8 T cell neo-antigens selected from the MC38 tumor model (vaccine, dose of 2×10\hat{ }7 vp), administered with Ad6-9d9 (10\hat{ }8 vp) mixed with the vaccine and injected in the mouse quadriceps, delivered as separate administration at the same anatomical sites or administered in two different anatomical sites (GAd vaccine in the left site and Ad-9d9 in the right site, contralateral). The same vaccine dose was used for all three regimes. As control, a group of mice received only the vaccine in absence of Ad-9d9. Shown are the immune responses (number of T cells producing IFNγ per millions of splenocytes) measured by an IFN-γ ELISpot assay.

FIG. 3 shows the effect of encoded anti-mCTLA4 antibody (Ad-9d9) on the vaccine-induced T cell response. A) BalBC mice were vaccinated intramuscularly (im) with a GAd vaccine encoding thirty-one CT26 neoantigens (vaccine) administered alone or in combination (mixture) with Ad6 encoding anti-mCTLA4 (Ad-9d9 dose of 10\hat{ }8 vp), or in combination with an anti-mCTLA4 antibody protein delivered intraperitoneally (ip) (9d9 Ab, 100 ug). Shown are the responses (number of T cells producing IFNγ per millions of splenocytes) to CD8 epitopes (light grey) and CD4 (dark grey) encoded in the vaccine measured in the 3 experimental groups (vaccine; vaccine+9d9 Ab; vaccine+Ad-9d9) by an IFN-γ ELISpot assay.

FIG. 4 shows the effect of encoded anti-mCTLA4 antibody (Ad6-9d9) on the vaccine-induced T cell response when co-administered with the vaccine, as well the impact of encoded anti-mCTLA4 on vaccine anti-tumor efficacy. A) BalBC mice were vaccinated intramuscularly (im) with a GAd vaccine encoding 62 CT26 neo-antigens (vaccine) administered alone or in combination (mixture) with Ad6 encoding anti-mCTLA4 (Ad-9d9 dose of 10\hat{ }8 vp). Shown are the immune responses (number of T cells producing IFNγ per millions of splenocytes) measured by an IFN-γ ELISpot assay. B) Antitumor efficacy of GAd-CT26-62 in combination with anti-mPD1 versus GAd-CT26-62 co-administered with Ad6-9d9 in combination with anti-mPD1. Treatments started at day 0, on mice randomized according to tumor volume. Tumor growth over time is shown for individual mice belonging to the 2 different groups of treatment. Anti-tumor response is evaluated as sum of complete and partial response (≥40% tumor shrinkage).

FIG. 5 shows the concentration of the anti-mCTLA4 antibody in the serum of injected mice. Shown is the anti-mCTLA4 antibody concentration in mice seven days post injection with Ad6 vector encoding the anti-mCTLA4 (Ad-9d9, black) or a single dose of anti-mCTLA4 antibody protein (9d9 Ab, 100 ug) injected subcutaneously (9d9 Ab sc, white) or ip (9d9 Ab ip, dark grey).

FIG. 6 shows the effect of encoded anti-CTLA4 to enhance the immunogenicity of a TAA based GAd vaccine. BalBC mice were vaccinated im with a GAd vaccine encoding 4 TAA selected from the CT26 tumors (vaccine, dose of 5×10\hat{ }8 vp), administered alone or in combination (mixture) with an Ad6 encoding anti-mCTLA4 (vaccine+Ad-9d9). Shown are the responses (number of T cells producing IFNγ per millions of splenocytes) to the encoded antigens (1 to 4) measured by using a set of peptides covering the vaccine sequence.

FIG. 7 shows the effect of encoded anti-mCTLA4 to enhance the antibody response against TAA. hHer2 transgenic (Tg) mice were vaccinated im with a GAd vaccine encoding hHer2 (Ad-hHer2, dose of 5×10\hat{ }8 vp), administered alone or in combination (mixture) with an Ad6 encoding anti-mCTLA4 (Ad-hHer2+Ad-9d9). 2 weeks after immunization, sera were prepared from immunized mice and analysed for the presence of Abs recognizing the TAA hHER2/neu. Sera from wt mice were used as a positive control, expected to be positive for a response against hHer2.

FIG. 8 shows the effect of encoded OX40L on the vaccine-induced T cell response. C57Bl6 mice were vaccinated im with a GAd vaccine encoding seven CD8 T cell neo-antigens selected from the MC38 tumor model (vaccine, dose of 2×10\hat{ }7 vp) administered alone or in combination (as mixture) with Ad encoding OX40L (Ad-OX40L, 10\hat{ }8 vp). As positive control, a group of mice received the vaccine in co-administration with Ad-9d9. Shown are the total responses (number of T cells producing IFNγ per millions of splenocytes) to the CD8 epitopes encoded in the vaccine measured by an IFN-γ ELISpot assay.

FIG. 9 shows the effect of encoded 9d9 and OX40L to break T cell tolerance to human Her2 in the hHer2 Tg mice. Mice were vaccinated im with a GAd vaccine encoding h-Her2 administered alone or in combination with an Adenovirus encoding either 9D9 (Ad-9D9) or OX40L (Ad-OX40L) or in combination with an equal mix of Ad-9D9 and Ad-OX40L. Shown are the T cell responses (number of T cells producing IFNγ per millions of splenocytes) to hHer2 measured by an IFN-γ ELISpot assay.

FIG. 10 shows the effect of encoded ICOSL on the vaccine-induced T cell response. C57Bl6 mice were vaccinated im with a GAd vaccine encoding seven CD8 T cell neo-antigens selected from the MC38 tumor model (vaccine, dose of 2×10\hat{ }7 vp) administered alone or in combination (as mixture) with Ad encoding ICOSL (Ad-ICOSL, 10\hat{ }8 vp). Shown are the total responses (number of T cells producing IFNγ per millions of splenocytes) to the CD8 epitopes encoded in the vaccine measured by an IFN-γ ELISpot assay.

FIG. 11 shows the impact of encoded Ad6 anti-mCTLA4 on vaccine anti-tumor efficacy in a regimen of single versus double administration of the adjuvant. Mice were inoculated s.c. with CT26 cells. One week later, animals were randomized according to tumor volume and treated at day 0 with the non adjuvanted vaccine GAd-CT26-62 in combination anti-PD1 (Vaccine+anti-PD1) versus the adjuvanted vaccine in a regimen of single administration of the encoded anti-CTLA4 (vaccine+Ad6-9d9+anti-PD1) or double administration (vaccine+Ad6-9d9 2×+anti-PD1) with the first dose of Ad6-9d9 coadministered with the vaccine at day 0, while the second given at day 1. Tumor growth over time is shown. Anti-tumor response is evaluated as sum of complete and partial response (≥40% tumor shrinkage).

FIG. 12 shows the serum concentration of the anti-hCTLA4 (Ipilimumab) in mice receiving Ad6-Ipi (10\hat{ }8 viral particles, vp) measured overtime by ELISA assay.

FIG. 13 shows the impact of a membrane-bound from of anti-mCTLA4 encoded in Ad6 (Ad6-9d9™) on vaccine anti-tumor efficacy. Shown are the total responses (number of T cells producing IFNγ per millions of splenocytes) to the CD8 epitopes encoded in the vaccine measured by an IFN-γ ELISpot assay, for the vaccine alone (Vaccine), Ad6-9d9 co-administered with the vaccine or Ad6-9d9™ co-administered with the vaccine.

### EXAMPLES

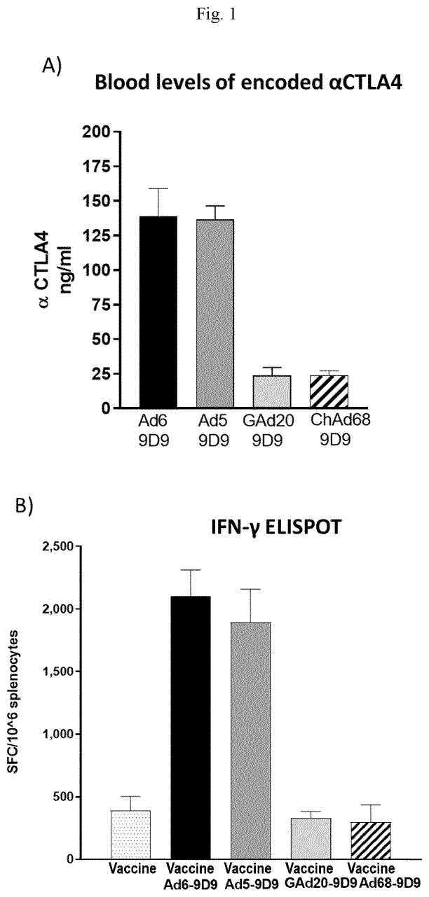

### Example 1: Ad-Encoded α-mCTLA4 Co-Administered Intramuscularly with an Adenoviral Vaccine Encoding Tumor Neoantigens Potentiates the Vaccine Induced T Cell Responses, but with Strongly Varying Efficiency (Ad6, Ad5>>GAd20 and ChAd68) (FIG. 1)

For this example, mice were vaccinated with a GAd vaccine encoding seven CD8 T cell neo-antigens selected from the MC38 tumor model (Yadav et al., Nature. 2014 Nov. 27; 515(7528):572-6; D'Alise et al, Nat. Commun. 2019 Jun. 19; 10(1):2688) injected alone or co-mixed with different adenoviral vectors encoding anti-mCTLA4 (clone 9d9, SEQ ID NO: 1) (Ad6-9d9; Ad5-9d9; GAd20-9d9; ChAd68-9d9). The adenoviral vectors encoding anti-mCTLA4 were administered at a dose of 10\hat{ }8 vp (10⁸ viral particles), which is equivalent to doses administered to human patients in clinical settings. Levels of circulating encoded anti-mCTLA4 were measured post Ad injection (day 7) in the different groups, showing higher level of the anti-mCTLA4 when encoded in Ad6 and Ad5 compared to GAd20 and ChAd68 (FIG. 1A). Immune responses were measured two weeks post vaccination by an ex-vivo IFN-γ ELISpot assay, using as antigens a pool of peptides corresponding to the sequence of each neoantigen present in the vaccine vector. Vaccine immunogenicity was enhanced in presence of encoded anti-mCTL4 expressed in Ad6 and Ad5 but not in GAd20 and ChAd68 (FIG. 1i).

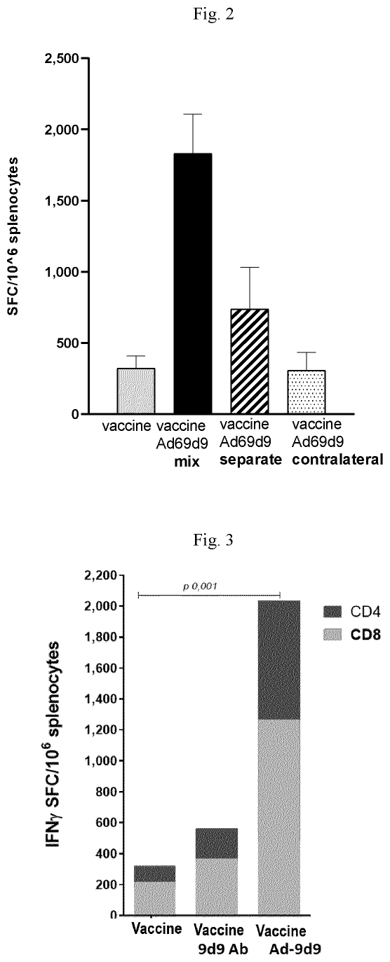

### Example 2: The Effect of Ad-Encoded α-mCTLA4 on Potentiating the Vaccine Induced T Cell Response Requires the Co-Administration with the Vaccine (FIG. 2)

To understand whether the effect of Ad-encoded α-mCTLA4 requires the coadministration with the vaccine as a mixture, C57Bl6 mice were vaccinated with a GAd vaccine encoding 7 CD8 neoantigens selected from the MC38 tumor model administered with Ad6-α-mCTLA4 in 3 different regimen modalities: i) in co-administration as a mixture in one anatomical site (quadriceps) ii) as two separate nearby administrations given within 5 min at the same anatomical site as i) and iii) as separate administrations at two contralateral distant sites. Immune responses were measured two weeks post vaccination by ex-vivo IFN-γ ELISpot assay, showing the loss of adjuvant effect when vaccine and Ad6-α-mCTLA4 were administered as separate components (FIG. 2). The adenoviral vector encoding the adjuvant α-mCTLA4 was administered at a dose of 10\hat{ }8 vp. The best effect on enhancing the immune response was achieved when vaccine and adjuvant Ad6-9d9 were co-administered as a mix.

### Example 3: Adenoviral Vector Encoded Anti-mCTLA4 Co-Administered Intramuscularly with an Adenoviral Vector Encoding Mouse Tumor Neoantigens Potentiates the Vaccine Induced T Cell Responses (CD8 and CD4) and Performs Better than the Same Antibody Systemically Delivered as Protein (FIG. 3)

For this example, mice were vaccinated with a polyneoantigen GAd vaccine encoding 31 neoantigens selected from CT26 murine tumors (D'Alise et al, Nat. Commun. 2019 Jun. 19; 10(1):2688). The vaccine was administered intramuscularly (10\hat{ }8 vp) alone or co-administered with Ad6-anti-mCTLA4 encoding an anti-mouse-CTLA4 (clone 9d9) at the dose of 10\hat{ }8 vp. A parallel group of mice was treated with the same vaccine in combination with the anti-mCTLA4 (clone 9d9) protein (BioXcell) given ip. Immune responses were measured two weeks later by ex-vivo IFN-γ ELISpot assay, by using as antigens a set of peptides corresponding to the sequence of each neoantigen present in the vaccine vector. Ad-encoded anti-mCTLA4 antibody co-administered with the GAd neoantigen vaccine increased both the vaccine-induced CD8+ and CD4+ T cell response against tumor neoantigens (FIG. 3). This effect was more potent than the one observed in presence of the anti-m-CTLA4 delivered as protein.

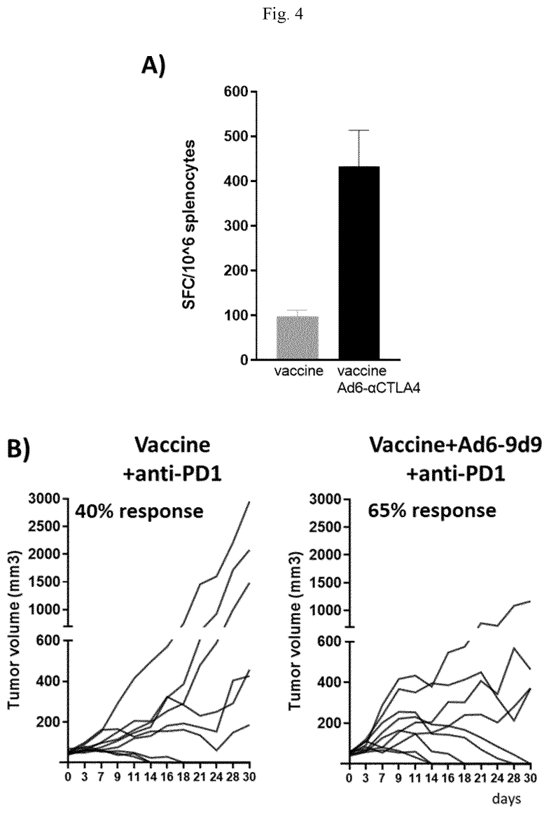

### Example 4: Adenoviral Vector Encoded Anti-CTLA4 Enhances Immune Response of a Genetic Vaccine Encoding 62 Neoantigens into Two Separate Expression Cassettes in Association with a Stronger Anti-Tumor Activity (FIG. 4)

The performance of the encoded adjuvant was also tested on a more complex construct encoding for a higher number of neoantigens (FIG. 4A). To this aim, a GAd vaccine vector encoding 62 neoantigens identified in the murine colon cancer cell line CT26 (named GAd-CT26-62) was used disclosed in (WO2020/099614 A1). Mice were vaccinated im with GAd-CT26-62 at a low dose (2×10\hat{ }7 vp), given alone or co-administered with Ad6-anti-CTLA4 encoding an anti-mouse anti-CTLA4 (clone 9D9) at the dose of 10\hat{ }8 vp. Immune responses were evaluated two weeks later by ex-vivo IFN-γ ELISpot assay, by using as antigens a set of peptides corresponding to the sequence of each neoantigen present in the vector. Adenovector-encoded anti-mCTLA4 antibody co-administered with GAd-CT26-62 increased the vaccine induced T cell response against tumor neoantigens (FIG. 4A). The same combination was also tested in the CT26 cancer mouse model to evaluate the impact of adenoviral vector encoded anti-CTLA4 co-administered with GAd vaccine on the anti-tumor activity in presence of anti-mPD1 treatment (clone RMP1-14 BioXcell), compared to the effect of the vaccine alone (no adjuvant) in presence of anti-mPD1.

The results showed enhanced antitumor activity of vaccine and anti-mPD1 when the vaccine was adjuvanted with the encoded Ad6-9d9 (FIG. 4B).

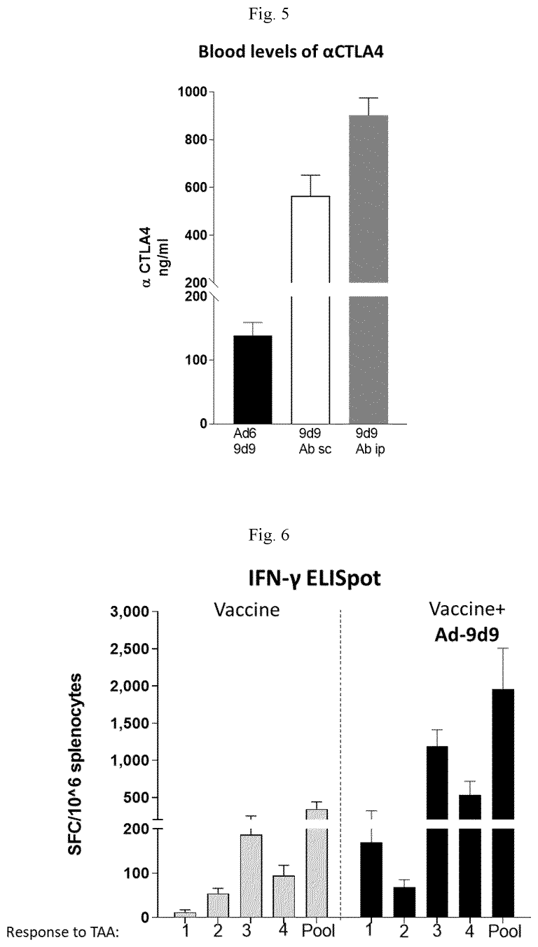

### Example 5: Limited Systemic Exposure to Anti-CTLA4 when Delivered by an Adenoviral Vector Compared to Systemic and Local Delivery of the Antibody Drug (FIG. 5)

For this example, mice were injected with Ad6 vector encoding an anti-mCTLA4 (Ad-9d9) at a dose of 10\hat{ }8 vp or a single dose of the same anti-mCTLA4 antibody given ip or sc (9d9 Ab, 100 ug). Measurement of the serum level of circulating anti-mCTLA4 after administration of the Ad6 demonstrated a very limited systemic exposure compared to the injection of the anti-mCTLA4 (clone 9D9 BioXcell) as protein, supporting improved biosafety for the encoded antibody (FIG. 5).

### Example 6: Adenoviral Vector Encoded Anti-CTLA4 Co-Administered Intramuscularly Together with an Adenoviral Vector Encoding Mouse Surrogate Tumor Associated Antigens (TAA) Breaks the Immune Tolerance (FIG. 6)

To interrogate the effect of adenoviral vector encoded anti-mCTLA4 in bypassing the immune tolerance of tumor associated antigens (TAA), the inventors selected surrogate TAA genes belonging to the family of antigens expressed in mouse CT26 tumors but not in healthy tissues. A vector encoding 4 murine TAAs (Slc9b1, Psg17, Gm773, Tcp11×2) preceded by a human tissue plasminogen activator (TPA) signal peptide was generated and injected in vivo alone or co-mixed with Ad6-encoded-anti-mCTLA4 at a dose of 10\hat{ }8 vp. Immune responses were measured two weeks post vaccination by ex-vivo IFN-γ ELISpot assay, by using as antigens a set of peptides corresponding to the sequence of the TAA encoded in the vaccine vector. Results showed a significant enhancement of the immune response when co-injecting Ad-9D9 together with the vaccine TAA (FIG. 6).

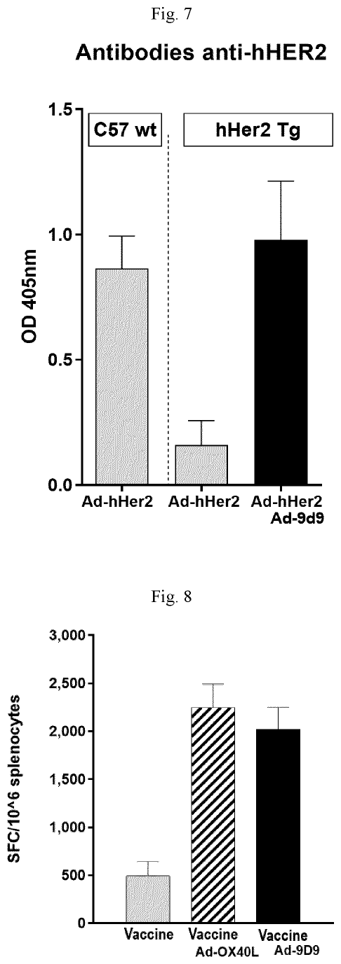

### Example 7: Adenoviral Vector Encoded Anti-CTLA4 Co-Administered Intramuscularly Together with an Adenoviral Vector Vaccine Encoding a Tumor Associated Antigen Also Increases the Antibody Response Versus a Self-Antigen (FIG. 7)

In this example, the effect of adenoviral vector encoded anti-mCTLA4 in also increasing the antibody response vaccine-induced was investigated. hHer2 transgenic (Tg) mice, a known mouse model tolerant to hHer2 and widely used to test Her2 vaccine, were immunized with a GAd vaccine encoding hHer2 injected alone or co-mixed with Ad6-9d9 at a dose of 10\hat{ }8 vp. Sera prepared from immunized mice were analyzed by ELISA against hHer2 protein to measure the antibody levels post treatment. Results showed that while the vaccine alone induces poor level of antibodies against hHer2, a relevant increase of the antibody response was observed in presence of encoded anti-mCTL4 expressed in Ad6.

### Example 8: Adenoviral Vector Encoded mOX40L Co-Administered with Ad Based Neoantigen Vaccine Enhances its Immunogenicity (FIG. 8)

For this example, mice were vaccinated with a GAd vaccine encoding seven CD8 T cell neo-antigens selected from the MC38 tumor model injected alone or co-mixed with adenovirus Ad6 encoding anti-mCTLA4 (Ad-9d9) and adenovirus Ad6 encoding mOX40L (Ad-OX40L). The adenoviral vectors encoding anti-mCTLA4 and mOX40L were administered at a dose of 10\hat{ }8 vp. Immune responses were measured two weeks post vaccination by ex-vivo IFN-γ ELISpot assay in each experimental group, using as antigens a pool of peptides corresponding to the sequence of each neoantigen present in the vaccine vector. Results show the potent effect of OX40L encoded in Ad6 in potentiating the vaccine immunogenicity, at similar levels of Ad-9d9.

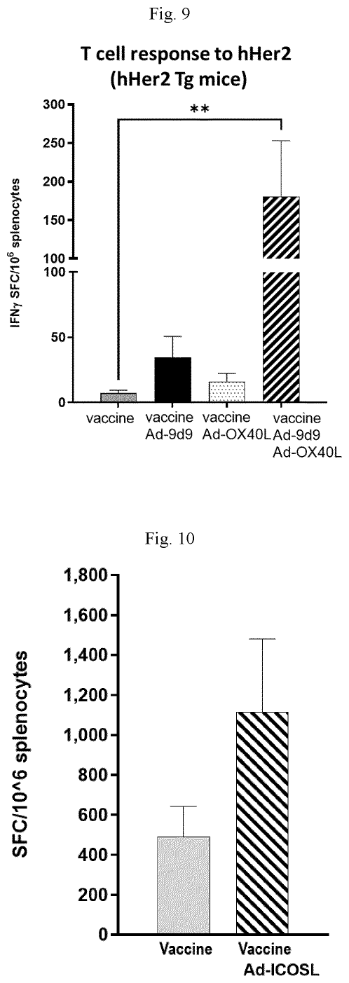

### Example 9: Use of the Two Encoded Adjuvants Anti-mCTLA4 and OX40L to Increase Vaccine Potency Against TAA in Stringent Mouse Model of T-Cell Tolerance (FIG. 9)

In this example, the effect of adenoviral vector encoded anti-mCTLA4 and Ad-OX40L was investigated in a stringent mouse model of T-cell tolerance against human Her2. hHer2 transgenic (Tg) mice, tolerant to hHer2, were immunized with a GAd vaccine encoding hHer2 injected alone, with a GAd vaccine encoding hHer2 co-mixed with either Ad6-9d9 or Ad6 OX40L at a dose of 10\hat{ }8 vp or with a GAd vaccine encoding hHer2 together with a mix of the two adjuvants. Immune responses were measured two weeks post vaccination by ex-vivo IFNγ ELISpot assay in each experimental group, showing the effect of the two encoded adjuvant in breaking T cell tolerance to human Her2 when both co-administered with the vaccine.

### Example 10: Adenoviral Vector Encoded ICOSL Co-Administered with Ad Based Neoantigen Vaccine Enhances its Immunogenicity (FIG. 10)

In this example, mice were vaccinated with a GAd vaccine encoding seven CD8 T cell neo-antigens selected from the MC38 tumor model injected alone or co-mixed with adenoviral Ad6 encoding murine ICOS-L (Ad-ICOSL) at a dose of 10\hat{ }8 vp. Immune responses were measured two weeks post vaccination by ex-vivo IFN-γ ELISpot assay in each experimental group, using as antigens a pool of peptides corresponding to the sequence of each neoantigen present in the vaccine vector. Results show enhancement of the vaccine-induced T cells responses by the encoded Ad-ICOSL.

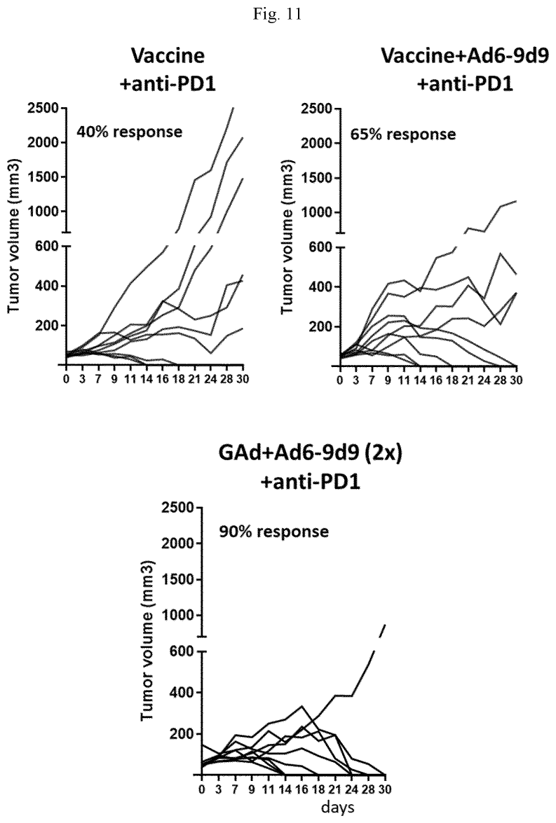

### Example 11: Re-Administration of Adenoviral Vector Encoded Anti-CTLA4 Enhances the Antitumor Efficacy of GAd Neoantigen Vaccine in Combination with Anti-PD1 (FIG. 11)

The impact of the encoded adjuvant anti-CTLA4 on the anti-tumor activity of GAd vaccine combined with a checkpoint inhibitor (anti-PD1) was tested in a regimen of single administration (vaccine plus Ad6-9d9, day 0) versus double administration (vaccine plus Ad6-9d9 at day 0; Ad6-9d9 at dl) in a mouse model of large established CT26 tumors. Tumor bearing mice were treated at day 0 with a GAd vaccine vector encoding 62 CT26 neoantigens (GAd-CT26-62) given alone or co-administered with Ad6-anti-CTLA4 encoding an anti-mCTLA4 (clone 9D9 10\hat{ }8 vp), in presence of anti-mPD1 (clone RMP1-14 BioXCell). A parallel group of mice received a second dose of Ad6-anti-CTLA4 the day after. The results showed enhanced antitumor activity of vaccine and anti-PD1 when the vaccine was adjuvanted with the encoded Ad6-9d9, with the best rate of 15 anti-tumor response observed in mice receiving two doses of Ad6-9d9.

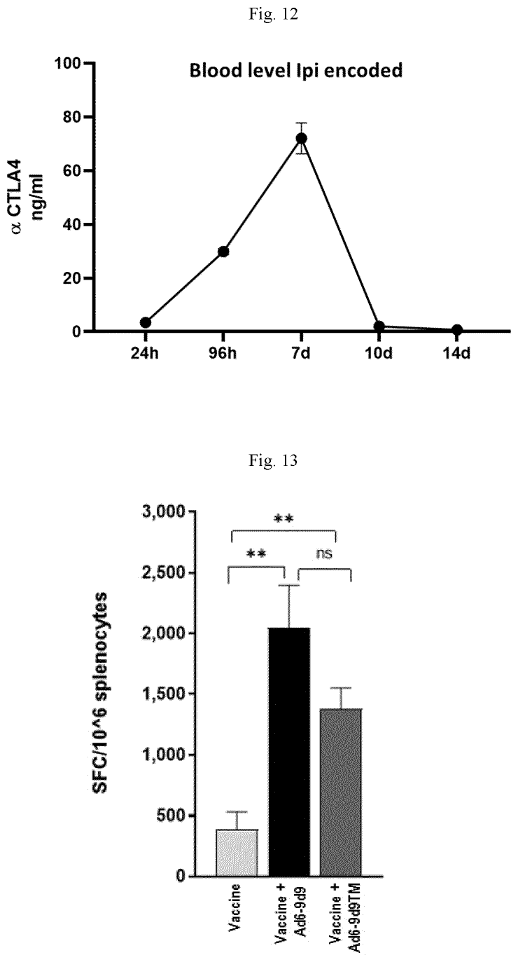

### Example 12: Measure of Circulating Anti-hCTLA4 in Mice after Injection with Ad6 Encoding Human Anti-CTLA4 (FIG. 12)

The sequence of anti-hCTLA4 Ipilimumab (SEQ ID NO: 2) was encoded in Ad6 and tested in vivo to evaluate its expression by Ad6. C57Bl6 mice were injected with Ad6-Ipilimumab at a dose of 10\hat{ }8 vp. The levels of circulating anti-hCTLA4 were measured post Ad injection over time, showing a detectable and good expression of the encoded Ipilimumab with a peak observed 7 days post Ad injection.

### Example 13: Ex-Vivo IFNγ ELISpot Assay

IFN-γ ELISpot assays were performed on single-cell suspensions of spleens. MSIP S4510 plates (Millipore, Billerica, MA) were coated with 10 μg/ml of anti-mouse IFN-γ antibody (Cat. Number: CT317-C; U-CyTech) and incubated overnight at 4° C. After washing and blocking the plates with media to avoid background, mouse splenocytes were plated in duplicate at two different cell densities and stimulated overnight with single 25-mer peptides or peptide pool at a final concentration of 1 g/ml. Peptide diluents dimethyl sulfoxide (Sigma-Aldrich) and concanavalin A (Sigma-Aldrich) were used, respectively, as negative and positive controls. Plates were developed by subsequent incubations with biotinylated anti-mouse IFN-γ antibody (dilution: 1/100; Cat. Number: CT317-D; U-CyTech), conjugated streptavidin-alkaline phosphatase (dilution: 1/2500; Cat. Number 554065; BD Biosciences) and finally with 5-bromo-4-chloro-3-indoyl-phosphate/nitro blue tetrazolium 1-Step solution (Thermo Fisher Scientific). An automated enzyme linked immunosorbent-spot assay video analysis system automated plate reader was used to analyze plates. ELISpot data were expressed as IFN-γ SFCs per million splenocytes. ELISpot responses were considered positive if all the following conditions occurred: (i) IFN-γ production present in ConA stimulated wells, (ii) the number of spots seen in positive wells was three times the number detected in the mock control wells (dimethyl sulfoxide), (iii) at least 30 specific spots/million splenocytes.

### Example 14: Adenoviral Vector Encoded Membrane-Bound Anti-CTLA4 Co-Administered with an Ad Based Neoantigen Vaccine Enhances Vaccine Immunogenicity

C57Bl6 mice were vaccinated i.m. with a GAd vaccine encoding seven CD8 T cell neo-antigens selected from the MC38 tumor model (vaccine, dose of 2×10\hat{ }7 vp) administered together with an Ad6 encoding a membrane-bound version of the 9d9 anti-mCTLA4 (Ad-9d9™), dose of 10\hat{ }8 vp) (SEQ ID NO: 3). Membrane-tethering was achieved by adding a transmembrane domain segment to the C-terminal end of the 9d9 heavy chain in SEQ ID NO: 2. As a positive control, a group of mice received the vaccine in co-administration with Ad6-9d9. Like the soluble form of 9d9, also the membrane-bound form enhanced vaccine immunogenicity as measured by an IFN-γ ELISpot assay (FIG. 13).

1. A vaccine composition comprising:
(1) a first set of one or more vectors comprising a nucleic acid encoding one or more adjuvants, wherein the first set of one or more vectors are adenoviral vectors; and
(2) an antigen or a combination of antigens or a nucleic acid encoding said antigen or combination of antigens or a second set of one or more vectors comprising said nucleic acid.

2. The vaccine composition according to claim 1, wherein the first set of one or more vectors are human adenoviral vectors.

3. The vaccine composition according to claim 2, wherein the human adenoviral vectors are selected from the group consisting of hAd6, hAd5 and hAd57, preferably selected from hAd6 and hAd57, more preferably hAd6.

4. The vaccine composition according to claim 1, wherein the antigen or combination of antigens is encoded by a nucleic acid that is not comprised in the first set of one or more vectors.

5. The vaccine composition according to claim 1, comprising a second set of one or more vectors comprising a nucleic acid encoding the antigen or combination of antigens, preferably wherein the second set of one or more vectors are adenoviral vectors, preferably derived from non-human Great Apes, more preferably derived from chimpanzee, or bonobo, or gorilla, most preferably derived from gorilla.

6. The vaccine composition according to claim 1, wherein the one or more adjuvants are selected from the group consisting of:
a. a modulator of an immune checkpoint molecule, preferably selected from the group consisting of:
an agonist of a tumor necrosis factor (TNF) receptor superfamily member or a B7-CD28 superfamily member, preferably an agonist of CD27, CD40, OX40, GITR, CD137, CD28 or ICOS, wherein preferably the agonist is a ligand or an agonistic antibody or antibody like protein (e.g. CP-870,893 for CD40); or
an antagonist of PD-1, PD-L1, A2AR, B7-H3 (e.g. MGA271), B7-H4, BTLA, CTLA-4, IDO, KIR, LAG3, TIM-3, TIGIT or VISTA, wherein preferably the antagonist is an (antagonistic) antibody or antibody like protein;
b. a cytokine, preferably IL-2, IL-1β, IL-7, IL-15, IL-18, GM-CFS, or INF-γ, and/or a cytokine analogue;
c. a cytokine receptor, preferably CD25 (IL-2 alpha receptor);
d. an activator of interferon (IFN) genes, preferably STING;
e. adenosine deaminase (ADA) or proliferator-activated receptor gamma coactivator 1-alpha (PGC-1α); and
f. a polynucleotide adjuvant.

7. The vaccine composition according to claim 1, wherein the one or more adjuvants are selected from the group consisting of an agonist of OX40, preferably OX40L, an agonist of ICOS, preferably ICOSL, an agonist of CD40, preferably CD40L and an antagonistic CTLA-4 specific antibody or antibody like protein, wherein the antagonistic CTLA-4 specific antibody or antibody like protein may be soluble or may comprise a transmembrane domain and an ER sorting signal.

8. The vaccine composition according to claim 1, wherein the one or more adjuvants comprise a transmembrane domain and an ER sorting signal.

9. The vaccine composition according to claim 1, wherein the antigen or the combination of antigens elicits no or only a suboptimal immune response in a subject in the absence of the adjuvant.

10. The vaccine composition according to claim 1, wherein the antigen or the combination of antigens comprises one or more cancer antigens selected from:
a. tumor associated antigens (TAAs), preferably TAAs-specific for a defined tumor type; and/or
b. cancer neo-antigens, preferably cancer neo-antigens selected from the group consisting-of a single amino acid mutant peptide, a frame-shift peptide, a read-through mutation peptide, and a splice site mutant peptide.

11. A method for the treatment or prophylaxis of a disease, comprising administering to a subject in need thereof the vaccine composition of claim 1 preferably for use in treating a proliferative disease, more preferably cancer, in a subject.

12. A vaccine composition or vaccine kit for inducing an immune response against an antigen or combination of antigens, comprising:
(1) a first composition comprising:
a first nucleic acid encoding one or more adjuvants, or
a first set of one or more vectors comprising said first nucleic acid; and
(2) a second composition comprising:
an antigen or a combination of antigens, or
a second nucleic acid encoding an antigen or a combination of antigens, or
a second set of one or more vectors comprising said second nucleic acid; wherein
a. (1) is administered to a patient at a first location and (2) is administered to the patient and at a second location, wherein the first location is within 20 cm, 17.5 cm, 15 cm, 12.5 cm, 10 cm, 7.5 cm, 5 cm, 2.5 cm, 1 cm, 0.5 cm, 0.25 cm or 0.1 cm of the second location and the lymphatic system of the first location drains to the same lymph nodes as the lymphatic system of the second location, or wherein the first location and the second location are the same; and optionally
b. the adjuvant comprises a transmembrane domain and an ER sorting signal.

13. The vaccine composition or vaccine kit of claim 12, wherein (1) and (2) are administered by intramuscular, subcutaneous, intradermal, intra-peritoneal or intra-pleural injection, wherein preferably, (1) and (2) are administered by the same route.

14. The vaccine composition or vaccine kit of claim 12, wherein (1) and (2) are administered within a time interval of 30 min or less, 20 min or less, 15 min or less, 10 min or less, 5 min or less, 3 min or less, or 1 min or less.

15. A vaccination regimen comprising a first and a second administration step, wherein
a. the first administration step comprises administration of a vaccine composition according to claim 1, and
b. the second administration step comprises administration of:
(1) a first composition comprising:
a first nucleic acid encoding one or more adjuvants, or
a first set of one or more vectors comprising said first nucleic acid; and/or
(2) a second composition comprising:
an antigen or a combination of antigens, or
a second nucleic acid encoding an antigen or a combination of antigens, or
a second set of one or more vectors comprising said second nucleic acid.
